(19) United States
(12) Patent Application Publication
(10) Pub. No.: US 2025/0154126 A1
(43) Pub. Date: May 15, 2025

(54) CRYSTALLINE FORM OF SULFUR-CONTAINING ISOINDOLINE DERIVATIVE

(71) Applicants: JIANGSU HENGRUI PHARMACEUTICALS CO., LTD., Lianyungang , Jiangsu (CN); SHANGHAI HENGRUI PHARMACEUTICAL CO., LTD, Shanghai (CN)

(72) Inventors: Baolei ZHANG, Shanghai (CN); Zhuoxun XI, Shanghai (CN); Jun FENG, Shanghai (CN); Feng He, Shanghai (CN); Junran YANG, Lianyungang (CN); Zhenxing DU, Lianyungang (CN)

(21) Appl. No.: 18/728,652

(22) PCT Filed: Jan. 19, 2023
(86) PCT No.: PCT/CN2023/073077

§ 371 Date: Jul. 12, 2024

(30) Foreign Application Priority Data
Jan. 19, 2022 (CN) 202210060175.6

(51) Int. Cl. C07D 401/14; A61K 31/4545; A61P 35/00

(52) U.S. Cl. CPC C07D 401/14; A61K 31/4545; A61P 35/00

(57) ABSTRACT

The present disclosure relates to a crystalline form of a sulfur-containing isoindoline derivative. Specifically, the present disclosure relates to a crystalline form of a compound as represented by formula (I) and a preparation method therefor.

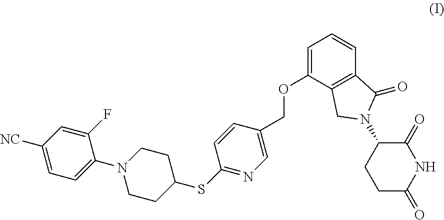 [SMILES: N#Cc1ccc(N2CCC(Sc3ccc(COc4cccc5c4CN([C@H]4CCC(=O)NC4=O)C5=O)cn3)CC2)c(F)c1]

The present application claims the right of the priority of Chinese patent application 202210060175.6 filed on Jan. 19, 2022. The content of the above Chinese patent application is incorporated herein by reference in its entirety.

### TECHNICAL FIELD

The present disclosure relates to a crystal form of a sulfur-containing isoindoline derivative and a preparation method therefor. Specifically, the present disclosure provides a crystal form of a compound of formula I and a preparation method therefor.

### BACKGROUND

Multiple myeloma (MM) is a malignant tumor, the main symptoms of which include hypercalcemia, renal injury, anemia, and bone disease. MM is the second most common hematological malignancy following non-Hodgkin's lymphoma. The current treatment methods are mainly drug therapy and autologous stem cell transplantation.

At present, there are four main classes of drugs widely used in clinical practice, which are alidomide immunomodulator, proteasome inhibitors, hormones and monoclonal antibodies. The drugs in clinical research stage include diabody, ADC, CAR-T, etc. Those drugs have different mechanisms of action, and they can often achieve better efficacy when used in combination. In clinical practice, dual, triple or even quadruple therapy is generally adopted, usually a combination therapy of an immunomodulator, a proteasome inhibitor and a hormone, sometimes with an additional antibody. Lenalidomide is the most commonly used immunomodulator, and is used in first-line therapy, maintenance therapy after stem cell transplantation, and second-line and third-line therapies after relapse. The drug reached $9.7 billion in sales in 2018/2019. In addition, the overall MM market is considerable and growing rapidly due to longer patient survival, and correspondingly longer duration of medication after continuous improvements and refinements in the diagnosis and treatment of MM. The MM market is expected to reach a scale of $33 billion in 2022, with the largest proportion still being immunomodulators such as lenalidomide.

The mechanism of action of immunomodulators (IMiD) for treating MM is mainly that IMiD drugs can activate the E3 ligase activity of CRBN after binding to Cereblon (CRBN) protein to selectively bind to transcription factors Ikaros (IKZF1) and Aiolos (IKZF3), thereby resulting in rapid ubiquitination and degradation of Ikaros and Aiolos. Downregulation of Ikaros/Aiolos results in downregulation of c-Myc, followed by the downregulation of IRF4, ultimately resulting in inhibition of growth and apoptosis of myeloma cells. In addition, IKZF3 can also inhibit the transcription of cytokines IL2 and TNF in T/NK cells. After the degradation of IKZF3, this inhibition can be relieved, and the release of these cytokines is promoted, so that an immunomodulatory effect is achieved. Clinical trials have also shown that the clinical benefit of IMiD drugs correlates with the expression level of CRBN. It was found that the inhibitory activity of lenalidomide for cell growth was lost and drug resistance was developed after CRBN was knocked down in lenalidomide-sensitive cell lines (OPM2 and KMS18), which indicates that the level of CRBN knock-down correlates with the degree of drug resistance. In a cell proliferation experiment, after the expression level of CRBN in cells (U266-CRBN60 and U266-CRBN75) was reduced, the inhibitory activity of both lenalidomide and pomalidomide for cell growth is reduced.

Currently, the IMiD drugs that have been approved for marketing include thalidomide, lenalidomide and pomalidomide, all from Celgene (now incorporated by BMS). For those three compounds, the binding forces to CRBN are increased sequentially, so the clinical doses are reduced sequentially. The primary indication for the three compounds is MM, and there are other indications for thalidomide and lenalidomide (especially lenalidomide), such as myelodysplastic syndrome (MDS). In terms of side effects, lenalidomide and pomalidomide are similar in performance, with significant myelosuppressive effect caused by target-related toxicity; thalidomide has some other side effects such as sedation, constipation, and neurological side effects.

The adipimide moiety of all IMiDs binds to a hydrophobic pocket defined by the three tryptophan residues in CRBN (referred to as “thalidomide binding pocket”). In contrast, the phthalimide/isoindolone ring is exposed to the solvent and alters the molecular surface of CRBN, thereby modulating substrate recognition. Different IMiDs result in significant modification of the surface of CRBN molecules and different preferences for substrate recognition. Thus, modifications of IMiDs may lead to degradation of other transcription factors, causing unwanted toxic and side effects. This mode of action of IMiDs is also known as molecular glue, which vividly expresses the bonding effect of this small molecule on two protein substrates.

Since the median survival for multiple myeloma is now more than five years, the prolonged survival leads to a high proportion of resistance to currently available drugs such as lenalidomide and pomalidomide in most patients, which seriously reduces the therapeutic effect of such drugs. Therefore, it is contemplated to develop more active drug molecules to overcome the problem of drug resistance whilst minimizing the toxic and side effects of such compounds.

Disclosed patent applications for Cereblon regulators include WO2008115516A2, WO2011100380A1, WO2019226770A1, WO2019014100A1, WO2020064002A1, etc.

The applicant's patent application WO2022017365 discloses a compound of formula I, chemically named (S)-4-(4-((5-(((2-(2,6-dioxopiperidin-3-yl)-1-oxoisoindolin-4-yl)oxy)methyl)pyridin-2-yl)thio)piperidin-1-yl)-3-fluorobenzonitrile. This compound exhibits a relatively good Cereblon modulating activity.

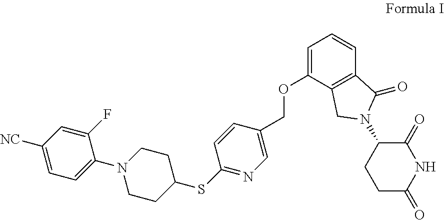 [SMILES: N#Cc1ccc(N2CCC(Sc3ccc(COc4cccc5c4CN([C@H]4CCC(=O)NC4=O)C5=O)cn3)CC2)c(F)c1]

Generally, the crystal forms of the active ingredients of a drug and its salts not only affect the physical and chemical stability of the drug itself, but also affect the difficulty of later drug preparation and production costs. Different crystallization and storage conditions may lead to changes in the crystal forms of the compound and its salts, sometimes resulting in the formation of other forms of crystal forms. Therefore, considering comprehensively from the perspectives of stability, ease of drug preparation process, production costs, etc., it is necessary to conduct an in-depth study of the crystal forms of the compound of formula (I).

### CONTENT OF THE PRESENT INVENTION

The present disclosure provides a crystal form of a compound of formula (I) and a preparation method therefor, wherein the compound of formula I is chemically named (S)-4-(4-((5-(((2-(2,6-dioxopiperidin-3-yl)-1-oxoisoindolin-4-yl)oxy)methyl)pyridin-2-yl)thio)piperidin-1-yl)-3-fluorobenzonitrile,

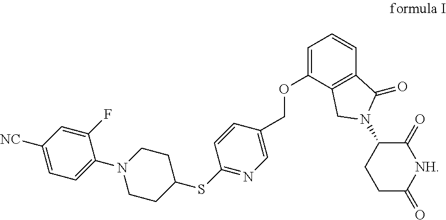 [SMILES: N#Cc1ccc(N2CCC(Sc3ccc(COc4cccc5c4CN([C@H]4CCC(=O)NC4=O)C5=O)cn3)CC2)c(F)c1]

The present disclosure provides an amorphous form of the compound of formula (I), which has an X-ray powder diffraction pattern comprising no obvious characteristic peaks at a diffraction angle 26 in the range of 2 to 48°.

The present disclosure further provides a method for preparing the amorphous form of the compound of formula (I), comprising method 1: a) mixing the compound of formula (I) with propylene glycol methyl ether, n-heptane, or petroleum ether; b) slurrying for crystallization.

In some embodiments, the volume (μl) of the solvent used in the present disclosure can be 1 to 200 times the mass (mg) of the compound of formula (I); in non-limiting embodiments, it can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200. In some embodiments, the method for preparing the amorphous form of the compound of formula (I) according to the present disclosure further includes steps of filtering, washing, or drying.

The present disclosure provides a crystal form A of the compound of formula (I), which has an X-ray powder diffraction pattern, represented by a diffraction angle 2θ, having characteristic peaks at 5.765, 8.061, 9.925, 16.632, 17.900, 19.469, and 21.115. In some embodiments, the crystal form A of the compound of formula (I) has characteristic peaks at 5.765, 7.465, 8.061, 9.925, 12.890, 15.085, 16.632, 17.900, 19.469, and 21.115. In some embodiments, the crystal form A of the compound of formula (I) has characteristic peaks at 5.765, 7.465, 8.061, 9.925, 11.674, 12.890, 14.270, 15.085, 16.632, 17.900, 18.715, 19.469, and 21.115. In some embodiments, the crystal form A of the compound of formula (I) has the X-ray powder diffraction pattern represented by the diffraction angle 2θ as shown in FIG. 2.

The present disclosure further provides a method for preparing the crystal form A of the compound of formula (I), comprising method 1: a) mixing the compound of formula (I) with tetrahydrofuran and dissolving to clarification; b) adding n-heptane for crystallization;
or method 2: a) mixing the compound of formula (I) with dichloromethane and dissolving to clarification; b) adding ethyl acetate for crystallization;
or method 3: a) mixing the compound of formula (I) with water, isopropyl acetate, acetone, ethyl acetate/ethanol, or ethyl acetate/n-heptane; b) slurrying for crystallization.

In some embodiments, the volume (μl) of the solvent used in the present disclosure can be 1 to 200 times the mass (mg) of the compound of formula (I); in non-limiting embodiments, it can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200. In some embodiments, the method for preparing the crystal form A of the compound of formula (I) according to the present disclosure further includes steps of filtering, washing, drying, etc.

The present disclosure provides a crystal form B of the compound of formula (I), which has an X-ray powder diffraction pattern, represented by a diffraction angle 2θ, having characteristic peaks at 4.977, 6.788, 10.047, 14.143, 15.684, 18.547, and 20.840. In some embodiments, the crystal form B of the compound of formula (I) has characteristic peaks at 4.977, 6.788, 10.047, 14.143, 15.684, 18.547, 20.840, 24.096, and 25.505. In some embodiments, the crystal form B of the compound of formula (I) has the X-ray powder diffraction pattern represented by the diffraction angle 2θ as shown in FIG. 3.

The present disclosure further provides a method for preparing the crystal form B of the compound of formula (I), comprising a) mixing the compound of formula (I) with ethyl acetate or acetonitrile/methanol; b) slurrying for crystallization, and drying at 30° C.

In some embodiments, the volume (μl) of the solvent used in the present disclosure can be 1 to 200 times the mass (mg) of the compound of formula (I); in non-limiting embodiments, it can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200. In some embodiments, the method for preparing the crystal form B of the compound of formula (I) according to the present disclosure further includes steps of filtering, washing, drying, etc.

The present disclosure provides a crystal form C of the compound of formula (I), which has an X-ray powder diffraction pattern, represented by a diffraction angle 2θ, having characteristic peaks at 5.653, 7.974, 9.989, 16.143, 17.860, 18.992, and 20.972. In some embodiments, the crystal form C of the compound of formula (I) has characteristic peaks at 5.653, 7.974, 9.989, 11.505, 12.798, 14.265, 16.143, 17.860, 18.992, and 20.972. In some embodiments, the crystal form C of the compound of formula (I) has characteristic peaks at 3.533, 5.653, 7.974, 8.790, 9.989, 11.505, 12.798, 14.265, 15.277, 16.143, 17.860, 18.992, and 20.972. In some embodiments, the crystal form C of the compound of formula (I) has the X-ray powder diffraction pattern represented by the diffraction angle 2θ as shown in FIG. 4.

The present disclosure further provides a method for preparing the crystal form C of the compound of formula (I), comprising method 1: a) mixing the compound of formula I with ethanol, isopropanol, methyl tert-butyl ether, methyl isobutyl ketone, water/ethanol, water/isopropanol, water/methanol (1:1), cyclohexane, or isopropyl ether; b) slurrying for crystallization;
or method 2: a) mixing the compound of formula (I) with dichloromethane, water/acetone, or tetrahydrofuran/ethanol, and dissolving to clarification; b) evaporating for crystallization.

In some embodiments, the volume (μl) of the solvent used in the present disclosure can be 1 to 200 times the mass (mg) of the compound of formula (I); in non-limiting embodiments, it can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200. In some embodiments, the method for preparing the crystal form C of the compound of formula (I) according to the present disclosure further includes steps of filtering, washing, drying, etc.

The present disclosure provides a crystal form D of the compound of formula (I), which has an X-ray powder diffraction pattern, represented by a diffraction angle 2θ, having characteristic peaks at 6.831, 9.845, 13.453, 18.225, 20.117, 20.891, and 23.006. In some embodiments, the crystal form D of the compound of formula (I) has characteristic peaks at 6.831, 9.845, 10.927, 13.453, 16.096, 18.225, 20.117, 20.891, 23.006, and 26.132. In some embodiments, the crystal form D of the compound of formula (I) has the X-ray powder diffraction pattern represented by the diffraction angle 2θ as shown in FIG. 5.

The present disclosure further provides a method for preparing the crystal form D of the compound of formula (I), comprising:
method 1: a) mixing the compound of formula (I) with acetonitrile; b) slurrying for crystallization.

In some embodiments, the volume (μl) of the solvent used in the present disclosure can be 1 to 200 times the mass (mg) of the compound of formula (I); in non-limiting embodiments, it can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200. In some embodiments, the method for preparing the crystal form D of the compound of formula (I) according to the present disclosure further includes steps of filtering, washing, drying, etc.

The present disclosure provides a crystal form E of the compound of formula (I), which has an X-ray powder diffraction pattern, represented by a diffraction angle 2θ, having characteristic peaks at 5.864, 7.573, 8.087, 10.003, 16.444, 19.349, and 20.553. In some embodiments, the crystal form E of the compound of formula (I) has characteristic peaks at 5.864, 7.573, 8.087, 10.003, 12.471, 15.165, 16.444, 17.432, 19.349, and 20.553. In some embodiments, the crystal form E of the compound of formula (I) has characteristic peaks at 5.864, 7.573, 8.087, 10.003, 11.701, 12.471, 15.165, 16.444, 17.432, 19.349, 20.553, 21.067, and 21.709. In some embodiments, the crystal form E of the compound of formula (I) has the X-ray powder diffraction pattern represented by the diffraction angle 2θ as shown in FIG. 6.

The present disclosure further provides a method for preparing the crystal form E of the compound of formula (I), comprising method 1: a) mixing the compound of formula I with 2-butanone or 10% water/methanol; b) slurrying for crystallization;
or method 2: a) mixing the compound of formula I with DCM and dissolving to clarification; b) adding n-heptane for crystallization.

In some embodiments, the volume (μl) of the solvent used in the present disclosure can be 1 to 200 times the mass (mg) of the compound of formula (I); in non-limiting embodiments, it can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200. In some embodiments, the method for preparing the crystal form E of the compound of formula (I) according to the present disclosure further includes steps of filtering, washing, drying, etc.

The present disclosure provides a crystal form F of the compound of formula (I), which has an X-ray powder diffraction pattern, represented by a diffraction angle 2θ, having characteristic peaks at 5.062, 7.820, 10.077, 14.231, 16.672, 18.586, and 20.435. In some embodiments, the crystal form F of the compound of formula (I) has characteristic peaks at 5.062, 7.820, 10.077, 14.231, 15.192, 16.672, 18.586, 20.435, 21.868, and 25.442. In some embodiments, the crystal form F of the compound of formula (I) has characteristic peaks at 5.062, 7.820, 10.077, 14.231, 15.192, 16.672, 18.586, 20.435, 21.868, 24.193, 25.442, 26.303, and 28.629. In some embodiments, the crystal form F of the compound of formula (I) has the X-ray powder diffraction pattern represented by the diffraction angle 2θ as shown in FIG. 7.

The present disclosure further provides a method for preparing the crystal form F of the compound of formula (I), comprising:
method 1: a) mixing the compound of formula (I) with a solvent II, wherein the solvent II is selected from p-xylene, methanol, 2-methyltetrahydrofuran, o-xylene, or toluene; b) slurrying for crystallization;
or method 2: a) mixing the compound of formula (I) with dichloromethane or tetrahydrofuran and dissolving to clarification; b) adding isopropyl acetate, methyl tert-butyl ether, or methyl isobutyl ketone for crystallization;
or method 3: a) mixing the compound of formula (I) with 1,4-dioxane and dissolving to clarification; b) evaporating for crystallization.

In some embodiments, the volume (μl) of the solvent used in the present disclosure can be 1 to 200 times the mass (mg) of the compound of formula (I); in non-limiting embodiments, it can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200. In some embodiments, the method for preparing the crystal form F of the compound of formula (I) according to the present disclosure further includes steps of filtering, washing, drying, etc.

The present disclosure provides a crystal form G of the compound of formula (I), which has an X-ray powder diffraction pattern, represented by a diffraction angle 2θ, having characteristic peaks at 5.999, 7.972, 9.951, 11.388, 17.812, 20.975, and 25.819. In some embodiments, the crystal form G of the compound of formula (I) has characteristic peaks at 5.999, 7.396, 7.972, 8.637, 9.951, 11.388, 15.291, 17.812, 20.975, and 25.819. In some embodiments, the crystal form G of the compound of formula (I) has characteristic peaks at 5.999, 7.396, 7.972, 8.637, 9.951, 11.388, 12.763, 15.291, 17.812, 20.975, 23.408, 25.819, and 27.400. In some embodiments, the crystal form G of the compound of formula (I) has the X-ray powder diffraction pattern represented by the diffraction angle 2θ as shown in FIG. 8.

The present disclosure further provides a method for preparing the crystal form G of the compound of formula (I), comprising:
method 1: a) mixing the compound of formula (I) with chloroform and dissolving to clarification; b) evaporating for crystallization;
or method 2: a) mixing the compound of formula (I) with 1,2-dichloroethane; b) slurrying for crystallization.

In some embodiments, the volume (μl) of the solvent used in the present disclosure can be 1 to 200 times the mass (mg) of the compound of formula (I); in non-limiting embodiments, it can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200. In some embodiments, the method for preparing the crystal form G of the compound of formula (I) according to the present disclosure further includes steps of filtering, washing, drying, etc.

The present disclosure provides a crystal form H of the compound of formula (I), which has an X-ray powder diffraction pattern, represented by a diffraction angle 2θ, having characteristic peaks at 5.758, 7.533, 9.901, 14.267, 16.420, 18.103, and 26.356. In some embodiments, the crystal form H of the compound of formula (I) has characteristic peaks at 5.758, 7.533, 9.901, 14.267, 16.420, 18.103, 18.917, 20.489, 24.049, and 26.356. In some embodiments, the crystal form H of the compound of formula (I) has characteristic peaks at 5.758, 7.533, 9.901, 14.267, 16.420, 18.103, 18.917, 20.489, 24.049, and 26.356. In some embodiments, the crystal form H of the compound of formula (I) has the X-ray powder diffraction pattern represented by the diffraction angle 2θ as shown in FIG. 9.

The present disclosure further provides a method for preparing the crystal form H of the compound of formula (I), comprising:
method 1: a) mixing the compound of formula (I) with n-octane or n-hexane; b) slurrying for crystallization.

In some embodiments, the volume (μl) of the solvent used in the present disclosure can be 1 to 200 times the mass (mg) of the compound of formula (I); in non-limiting embodiments, it can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200. In some embodiments, the method for preparing the crystal form H of the compound of formula (I) according to the present disclosure further includes steps of filtering, washing, drying, etc.

The present disclosure provides a crystal form I of the compound of formula (I), which has an X-ray powder diffraction pattern, represented by a diffraction angle 2θ, having characteristic peaks at 5.294, 6.826, 7.564, 10.739, 13.699, 16.812, and 20.709. In some embodiments, the crystal form I of the compound of formula (I) has the X-ray powder diffraction pattern represented by the diffraction angle 2θ as shown in FIG. 10.

The present disclosure further provides a method for preparing the crystal form I of the compound of formula (I), comprising:
method 1: a) mixing the compound of formula (I) with tetrahydrofuran and dissolving to clarification; b) evaporating for crystallization;
or method 2: a) mixing the compound of formula (I) with tetrahydrofuran and dissolving to clarification; b) adding water for crystallization.

In some embodiments, the volume (μl) of the solvent used in the present disclosure can be 1 to 200 times the mass (mg) of the compound of formula (I); in non-limiting embodiments, it can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200. In some embodiments, the method for preparing the crystal form I of the compound of formula (I) according to the present disclosure further includes steps of filtering, washing, drying, etc.

The present disclosure provides a crystal form J of the compound of formula (I), which has an X-ray powder diffraction pattern, represented by a diffraction angle 2θ, having characteristic peaks at 5.041, 10.068, 16.424, 20.544, 21.190, 24.077, and 25.433. In some embodiments, the crystal form J of the compound of formula (I) has characteristic peaks at 5.041, 8.212, 10.068, 14.101, 15.167, 16.424, 20.544, 21.190, 24.077, and 25.433. In some embodiments, the crystal form J of the compound of formula (I) has characteristic peaks at 5.041, 8.212, 10.068, 14.101, 15.167, 16.424, 20.544, 21.190, 22.036, 22.679, 24.077, 25.433, and 26.454. In some embodiments, the crystal form J of the compound of formula (I) has the X-ray powder diffraction pattern represented by the diffraction angle 2θ as shown in FIG. 11.

The present disclosure further provides a method for preparing the crystal form J of the compound of formula (I), comprising: a) mixing the compound of formula (I) with dimethyl sulfoxide and dissolving to clarification; b) adding water, isopropyl acetate, or methyl tert-butyl ether for crystallization.

In some embodiments, the volume (μl) of the solvent used in the present disclosure can be 1 to 200 times the mass (mg) of the compound of formula (I); in non-limiting embodiments, it can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200. In some embodiments, the method for preparing the crystal form J of the compound of formula (I) according to the present disclosure further includes steps of filtering, washing, drying, etc.

The present disclosure provides a crystal form K of the compound of formula (I), which has an X-ray powder diffraction pattern, represented by a diffraction angle 2θ, having characteristic peaks at 5.156, 7.699, 10.339, 14.334, 16.203, 18.327, and 23.418. In some embodiments, the crystal form K of the compound of formula (I) has characteristic peaks at 5.156, 7.699, 10.339, 14.334, 16.203, 18.327, 23.418, 25.348, 25.919, and 26.446. In some embodiments, the crystal form K of the compound of formula (I) has the X-ray powder diffraction pattern represented by the diffraction angle 2θ as shown in FIG. 12.

The present disclosure further provides a method for preparing the crystal form K of the compound of formula (I), comprising: a) mixing the compound of formula (I) with N-methylpyrrolidone and dissolving to clarification; b) evaporating for crystallization.

In some embodiments, the volume (μl) of the solvent used in the present disclosure can be 1 to 200 times the mass (mg) of the compound of formula (I); in non-limiting embodiments, it can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200. In some embodiments, the method for preparing the crystal form K of the compound of formula (I) further includes steps of filtering, washing, drying, etc.

The present disclosure provides a crystal form L of the compound of formula (I), which has an X-ray powder diffraction pattern, represented by a diffraction angle 2θ, having characteristic peaks at 4.932, 5.360, 9.831, 14.844, 18.244, 20.104, and 24.914. In some embodiments, the crystal form L of the compound of formula (I) has characteristic peaks at 4.932, 5.360, 9.831, 10.753, 14.844, 16.369, 18.244, 20.104, 23.129, and 24.914. In some embodiments, the crystal form L of the compound of formula (I) has the X-ray powder diffraction pattern represented by the diffraction angle 2θ as shown in FIG. 13.

The present disclosure further provides a method for preparing the crystal form L of the compound of formula (I), comprising: a) mixing the compound of formula (I) with N,N-dimethylacetamide and dissolving to clarification; b) evaporating for crystallization.

In some embodiments, the volume (μl) of the solvent used in the present disclosure can be 1 to 200 times the mass (mg) of the compound of formula (I); in non-limiting embodiments, it can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200. In some embodiments, the method for preparing the crystal form L of the compound of formula (I) according to the present disclosure further includes steps of filtering, washing, drying, etc.

The present disclosure provides a crystal form M of the compound of formula (I), which has an X-ray powder diffraction pattern, represented by a diffraction angle 2θ, having characteristic peaks at 14.959, 16.322, 18.410, 20.748, 22.067, 23.670, and 26.863. In some embodiments, the crystal form M of the compound of formula (I) has characteristic peaks at 14.959, 16.322, 18.410, 20.748, 22.067, 23.670, 24.839, 25.873, 26.863, and 27.811. In some embodiments, the crystal form M of the compound of formula (I) has characteristic peaks at 14.959, 16.322, 18.410, 20.748, 22.067, 23.670, 24.322, 24.839, 25.873, 26.863, and 27.811. In some embodiments, the crystal form M of the compound of formula (I) has the X-ray powder diffraction pattern represented by the diffraction angle 2θ as shown in FIG. 14.

The present disclosure further provides a method for preparing the crystal form M of the compound of formula (I), comprising: a) heating the crystal form A, crystal form B, crystal form C, crystal form E, crystal form H, or crystal form I of the compound of formula (I) to 225° C.

In some embodiments, the volume (μl) of the solvent used in the present disclosure can be 1 to 200 times the mass (mg) of the compound of formula (I); in non-limiting embodiments, it can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200. In some embodiments, the method for preparing the crystal form M of the compound of formula (I) according to the present disclosure further includes steps of filtering, washing, drying, etc.

The present disclosure provides a crystal form N of the compound of formula (I), which has an X-ray powder diffraction pattern, represented by a diffraction angle 2θ, having characteristic peaks at 5.028, 9.942, 10.900, 15.428, 18.410, 20.274, and 25.252. In some embodiments, the crystal form N of the compound of formula (I) has characteristic peaks at 5.028, 7.671, 9.942, 10.900, 15.428, 16.560, 18.410, 20.274, 24.036, and 25.252. In some embodiments, the crystal form N of the compound of formula (I) has characteristic peaks at 5.028, 7.671, 9.942, 10.900, 12.677, 15.428, 16.560, 18.410, 20.274, 24.036, 25.252, and 26.385. In some embodiments, the crystal form N of the compound of formula (I) has the X-ray powder diffraction pattern represented by the diffraction angle 2θ as shown in FIG. 15.

The present disclosure further provides a method for preparing the crystal from N of the compound of formula (I), comprising: a) mixing the compound of formula (I) with ethyl acetate/tetrahydrofuran (1:1), heating and dissolving to clarification; b) cooling for crystallization.

In some embodiments, the volume (μl) of the solvent used in the present disclosure can be 1 to 200 times the mass (mg) of the compound of formula (I); in non-limiting embodiments, it can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200. In some embodiments, the method for preparing the crystal form N of the compound of formula (I) according to the present disclosure further includes steps of filtering, washing, drying, etc.

The present disclosure provides a crystal form O of the compound of formula (I), which has an X-ray powder diffraction pattern, represented by a diffraction angle 2θ, having characteristic peaks at 11.796, 17.423, 18.081, 19.136, 21.707, 22.165, and 25.719. In some embodiments, the crystal form O of the compound of formula (I) has characteristic peaks at 7.843, 11.796, 17.423, 18.081, 19.136, 21.707, 22.165, 24.412, 25.719, and 28.521. In some embodiments, the crystal form O of the compound of formula (I) has characteristic peaks at 7.843, 11.796, 15.455, 17.423, 18.081, 19.136, 21.055, 21.707, 22.165, 24.412, 25.719, 27.538, and 28.521. In some embodiments, the crystal form O of the compound of formula (I) has the X-ray powder diffraction pattern represented by the diffraction angle 2θ as shown in FIG. 16.

The present disclosure further provides a method for preparing the crystal from O of the compound of formula (I), comprising: a) mixing the compound of formula I with acetonitrile and benzenesulfonic acid, and heating; b) cooling for crystallization.

In some embodiments, the volume (μl) of the solvent used in the present disclosure can be 1 to 200 times the mass (mg) of the compound of formula (I); in non-limiting embodiments, it can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200. In some embodiments, the method for preparing the crystal form O of the compound of formula (I) according to the present disclosure further includes steps of filtering, washing, drying, etc.

The present disclosure provides a crystal form P of the compound of formula (I), which has an X-ray powder diffraction pattern, represented by a diffraction angle 2θ, having characteristic peaks at 5.359, 7.491, 10.786, 14.249, 16.527, 17.729, and 20.798. In some embodiments, the crystal form P of the compound of formula (I) has characteristic peaks at 5.359, 7.491, 9.905, 10.786, 13.192, 14.249, 16.527, 17.729, 18.862, and 20.798. In some embodiments, the crystal form P of the compound of formula (I) has characteristic peaks at 5.359, 7.491, 9.905, 10.786, 13.192, 14.249, 16.527, 17.729, 18.862, 20.798, 23.799, and 26.555. In some embodiments, the crystal form P of the compound of formula (I) has the X-ray powder diffraction pattern represented by the diffraction angle 2θ as shown in FIG. 17.

The present disclosure further provides a method for preparing the crystal form P of the compound of formula (I), comprising: a) mixing the crystal form C of the compound of formula (I) with 1,4-dioxane; b) slurrying for crystallization.

In some embodiments, the volume (μl) of the solvent used in the present disclosure can be 1 to 200 times the mass (mg) of the compound of formula (I); in non-limiting embodiments, it can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200. In some embodiments, the method for preparing the crystal form P of the compound of formula (I) according to the present disclosure further includes steps of filtering, washing, drying, etc.

The present disclosure provides a crystal form Q of the compound of formula (I), which has an X-ray powder diffraction pattern, represented by a diffraction angle 2θ, having characteristic peaks at 5.038, 10.152, 15.850, 16.574, 18.892, 20.760, and 21.835. In some embodiments, the crystal form Q of the compound of formula (I) has characteristic peaks at 5.038, 10.152, 11.175, 15.850, 16.574, 18.892, 20.760, 21.835, 23.905, and 25.784. In some embodiments, the crystal form Q of the compound of formula (I) has characteristic peaks at 5.038, 7.682, 10.152, 11.175, 14.218, 15.850, 16.574, 18.892, 20.760, 21.835, 23.905, 25.784, and 26.418. In some embodiments, the crystal form Q of the compound of formula (I) has the X-ray powder diffraction pattern represented by the diffraction angle 2θ as shown in FIG. 18.

The present disclosure further provides a method for preparing the crystal form Q of the compound of formula (I), comprising method 1: a) mixing the compound of formula (I) with ethyl acetate; b) slurrying for crystallization, and drying the solid at 70° C. or 130° C.

In some embodiments, the volume (μl) of the solvent used in the present disclosure can be 1 to 200 times the mass (mg) of the compound of formula (I); in non-limiting embodiments, it can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200. In some embodiments, the method for preparing the crystal form Q of the compound of formula (I) according to the present disclosure further includes steps of filtering, washing, drying, etc.

The present disclosure provides a crystal form U of the compound of formula (I), which has an X-ray powder diffraction pattern, represented by a diffraction angle 2θ, having characteristic peaks at 14.155, 15.745, 17.314, 17.997, 18.838, 20.512, and 21.415. In some embodiments, the crystal form U of the compound of formula (I) has characteristic peaks at 14.155, 15.745, 16.564, 17.314, 17.997, 18.838, 20.512, 21.415, 23.557, and 26.313. In some embodiments, the crystal form U of the compound of formula (I) has characteristic peaks at 7.657, 14.155, 15.745, 16.564, 17.314, 17.997, 18.838, 20.512, 21.415, 23.557, 25.711, 26.313, and 28.029. In some embodiments, the crystal form U of the compound of formula (I) has the X-ray powder diffraction pattern represented by the diffraction angle 2θ as shown in FIG. 19.

The present disclosure further provides a method for preparing the crystal form U of the compound of formula (I), comprising method 1: a) mixing the crystal form Q of the compound of formula (I) with water; b) slurrying for crystallization;
or method 2: a) mixing the compound of formula I with DMSO, heating and dissolving to clarification; b) adding water for crystallization.

In some embodiments, the volume (μl) of the solvent used in the present disclosure can be 1 to 200 times the mass (mg) of the compound of formula (I); in non-limiting embodiments, it can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200. In some embodiments, the method for preparing the crystal form U of the compound of formula (I) according to the present disclosure further includes steps of filtering, washing, drying, etc.

The present disclosure provides a crystal form X of the compound of formula (I), which has an X-ray powder diffraction pattern, represented by a diffraction angle 2θ, having characteristic peaks at 6.864, 9.873, 10.963, 13.801, 16.089, 18.006, and 20.929. In some embodiments, the crystal form X of the compound of formula (I) has characteristic peaks at 6.864, 9.873, 10.963, 13.801, 16.089, 18.006, 20.929, and 26.203. In some embodiments, the crystal form X of the compound of formula (I) has the X-ray powder diffraction pattern represented by the diffraction angle 2θ as shown in FIG. 20.

The present disclosure further provides a method for preparing the crystal form X of the compound of formula (I): comprising: method 1: a) mixing the crystal form D of the compound of formula (I) with isopropyl ether or n-heptane; b) slurrying for crystallization.

In some embodiments, the volume (μl) of the solvent used in the present disclosure can be 1 to 200 times the mass (mg) of the compound of formula (I); in non-limiting embodiments, it can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200. In some embodiments, the method for preparing the crystal form X of the compound of formula (I) according to the present disclosure further includes steps of filtering, washing, drying, etc.

The present disclosure provides a crystal form Y of the compound of formula (I), which has an X-ray powder diffraction pattern, represented by a diffraction angle 2θ, having characteristic peaks at 5.676, 7.666, 14.260, 16.562, 18.020, 21.802, and 26.425. In some embodiments, the crystal form Y of the compound of formula (I) has characteristic peaks at 5.676, 7.666, 9.985, 12.634, 14.260, 16.562, 18.020, 21.802, 26.425, and 26.974. In some embodiments, the crystal form Y of the compound of formula (I) has characteristic peaks at 5.676, 7.666, 9.985, 12.634, 14.260, 16.562, 18.020, 21.802, 24.051, 25.846, 26.425, and 26.974. In some embodiments, the crystal form Y of the compound of formula (I) has the X-ray powder diffraction pattern represented by the diffraction angle 2θ as shown in FIG. 21.

The present disclosure further provides a method for preparing the crystal form Y of the compound of formula (I): comprising: a) mixing the crystal form Q of the compound of formula (I) with isopropyl ether or n-heptane; b) slurrying for crystallization.

In some embodiments, the volume (μl) of the solvent used in the present disclosure can be 1 to 200 times the mass (mg) of the compound of formula (I); in non-limiting embodiments, it can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200. In some embodiments, the method for preparing the crystal form Y of the compound of formula (I) according to the present disclosure further includes steps of filtering, washing, drying, etc.

The present disclosure provides a crystal form V of the compound of formula (I), which has an X-ray powder diffraction pattern, represented by a diffraction angle 2θ, having characteristic peaks at 5.649, 6.154, 6.720, 11.651, 18.757, 19.813, and 23.948. In some embodiments, the crystal form V of the compound of formula (I) has characteristic peaks at 5.649, 6.154, 6.720, 9.778, 11.651, 17.570, 18.757, 19.813, 23.948, and 26.995. In some embodiments, the crystal form V of the compound of formula (I) has characteristic peaks at 5.649, 6.154, 6.720, 9.778, 11.651, 13.576, 17.570, 18.757, 19.813, 21.905, 23.948, 25.825, and 26.995. In some embodiments, the crystal form V of the compound of formula (I) has the X-ray powder diffraction pattern represented by the diffraction angle 2θ as shown in FIG. 22.

The present disclosure further provides a method for preparing the crystal form V of the compound of formula (I), comprising method 1: a) mixing the compound of formula (I) with N,N-dimethylformamide and dissolving to clarification; b) adding acetone or acetonitrile for crystallization;
or method 2: a) mixing the compound of formula (I) with N,N-dimethylformamide; b) cooling for crystallization or stirring for crystallization.

In some embodiments, the volume (μl) of the solvent used in the present disclosure can be 1 to 200 times the mass (mg) of the compound of formula (I); in non-limiting embodiments, it can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200. In some embodiments, the method for preparing the crystal form V of the compound of formula (I) according to the present disclosure further includes steps of filtering, washing, drying, etc.

The present disclosure provides a crystal form R of the compound of formula (I), which has an X-ray powder diffraction pattern, represented by a diffraction angle 2θ, having characteristic peaks at 5.534, 7.611, 10.033, 15.782, 17.101, 19.017, and 20.567. In some embodiments, the crystal form R of the compound of formula (I) has characteristic peaks at 5.534, 7.611, 10.033, 11.857, 12.737, 15.782, 17.101, 19.017, 20.567, and 23.692. In some embodiments, the crystal form R of the compound of formula (I) has characteristic peaks at 5.534, 7.611, 10.033, 11.148, 11.857, 12.737, 14.179, 15.782, 17.101, 19.017, 20.567, 21.871, and 23.692. In some embodiments, the crystal form R of the compound of formula (I) has the X-ray powder diffraction pattern represented by the diffraction angle 2θ as shown in FIG. 23.

The present disclosure further provides a method for preparing the crystal form R of the compound of formula (I), comprising: a) mixing the compound of formula (I) with tetrahydrofuran and dissolving; b) concentrating for crystallization.

In some embodiments, the volume (μl) of the solvent used in the present disclosure can be 1 to 200 times the mass (mg) of the compound of formula (I); in non-limiting embodiments, it can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200. In some embodiments, the method for preparing the crystal form R of the compound of formula (I) according to the present disclosure further includes steps of filtering, washing, drying, etc.

The present disclosure provides a crystal form S of the compound of formula (I), which has an X-ray powder diffraction pattern, represented by a diffraction angle 2θ, having characteristic peaks at 6.576, 9.082, 10.921, 13.592, 19.965, 21.403, and 24.207. In some embodiments, the crystal form S of the compound of formula (I) has characteristic peaks at 6.576, 9.082, 10.921, 13.592, 16.805, 19.965, 21.403, 24.207, 25.662, and 27.457. In some embodiments, the crystal form S of the compound of formula (I) has characteristic peaks at 6.576, 7.890, 9.082, 10.921, 13.592, 15.043, 16.805, 19.965, 21.403, 24.207, 25.662, 26.537, and 27.457. In some embodiments, the crystal form S of the compound of formula (I) has the X-ray powder diffraction pattern represented by the diffraction angle 2θ as shown in FIG. 24.

The present disclosure further provides a method for preparing the crystal form S of the compound of formula (I), comprising method 1: a) mixing the compound of formula (I) with DMF, heating to dissolve, and cooling to precipitate; b) filtering, mixing the filter cake with acetonitrile, and slurrying; c) filtering, mixing the filter cake with water, and slurrying; d) filtering, drying, then slurrying with acetonitrile for crystallization, and drying.

In some embodiments, the volume (μl) of the solvent used in the present disclosure can be 1 to 200 times the mass (mg) of the compound of formula (I); in non-limiting embodiments, it can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200. In some embodiments, the method for preparing the crystal form S of the compound of formula (I) according to the present disclosure further includes steps of filtering, washing, drying, etc.

The present disclosure provides a crystal form T of the compound of formula (I), which has an X-ray powder diffraction pattern, represented by a diffraction angle 2θ, having characteristic peaks at 6.915, 9.177, 9.984, 11.012, 13.595, 16.156, and 20.138. In some embodiments, the crystal form T of the compound of formula (I) has characteristic peaks at 6.915, 9.177, 9.984, 11.012, 13.595, 15.174, 16.156, 20.138, 24.261, and 26.391. In some embodiments, the crystal form T of the compound of formula (I) has characteristic peaks at 6.915, 9.177, 9.984, 11.012, 13.595, 15.174, 16.156, 18.509, 20.138, 22.954, 24.261, 26.391, and 27.514. In some embodiments, the crystal form T of the compound of formula (I) has the X-ray powder diffraction pattern represented by the diffraction angle 2θ as shown in FIG. 25.

The present disclosure further provides a method for preparing the crystal form T of the compound of formula (I), comprising method 1: a) mixing the compound of formula (I) with DMF, heating to dissolve, and cooling for crystallization; b) filtering, mixing the filter cake with water and stirring; c) filtering, again mixing the filter cake with water and stirring; d) filtering, drying, then mixing with acetonitrile, stirring for crystallization, and drying.

In some embodiments, the volume (μl) of the solvent used in the present disclosure can be 1 to 200 times the mass (mg) of the compound of formula (I); in non-limiting embodiments, it can be 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200. In some embodiments, the method for preparing the crystal form T of the compound of formula (I) according to the present disclosure further includes steps of filtering, washing, drying, etc.

The present disclosure further provides a pharmaceutical composition prepared from the aforementioned crystal forms of the compound of formula (I).

The present disclosure further provides a pharmaceutical composition comprising the aforementioned crystal form, and optionally a pharmaceutically acceptable carrier, a diluent, or an excipient.

The present disclosure further provides a method for preparing a pharmaceutical composition, the method comprising the step of mixing the aforementioned crystal form with a pharmaceutically acceptable carrier, a diluent, or an excipient.

The present disclosure further provides a use of the aforementioned crystal form, the aforementioned composition, or the composition prepared by the aforementioned method in the manufacture of a medicament for treating and/or preventing a CRBN protein-related disease.

The present disclosure further provides a use of the aforementioned crystal form, the aforementioned composition, or the composition prepared by the aforementioned method in the manufacture of a medicament for treating and/or preventing a cancer, an angiogenesis-related condition, pain, macular degeneration or related syndrome, a skin disease, a pulmonary disease, an asbestos-related disease, a parasitic disease, an immunodeficiency disease, a CNS disease, a CNS injury, atherosclerosis or related condition, sleep disorder or related condition, an infectious disease, hemoglobinopathy or related condition, or a TNFα-related condition; preferably, a use in the manufacture of a medicament for treating and/or preventing a cancer or a CNS injury.

In some embodiments, the cancer is selected from the group consisting of leukemia, myeloma, lymphoma, melanoma, skin cancer, liver cancer, kidney cancer, lung cancer, nasopharyngeal cancer, gastric cancer, esophageal cancer, colorectal cancer, gallbladder cancer, bile duct cancer, chorionic epithelioma, pancreatic cancer, polycythemia vera, pediatric tumor, cervical cancer, ovarian cancer, breast cancer, bladder cancer, urothelial cancer, ureteral tumor, prostate cancer, seminoma, testicular tumor, head and neck tumor, head and neck squamous cell carcinoma, endometrial cancer, thyroid cancer, sarcoma, osteoma, neuroblastoma, neuroendocrine cancer, brain tumor, CNS cancer, astrocytoma, and glioma; preferably, the liver cancer is hepatocellular carcinoma; the colorectal cancer is colon cancer or rectal cancer; the sarcoma is osteosarcoma or soft tissue sarcoma; and the glioma is glioblastoma.

In some embodiments, the myeloma is multiple myeloma (MM) and myelodysplastic syndrome (MDS); preferably, the multiple myeloma is relapsed, refractory, or resistant.

In some embodiments, the multiple myeloma is refractory or resistant to lenalidomide or pomalidomide.

The “2θ” or “2θ angle” in the present disclosure refers to the diffraction angle, where θ represents the Bragg angle with units in ° or degrees. The error range of 2θ for each characteristic peak is ±0.20 (including cases where numbers exceeding one decimal place are rounded), and may be −0.20, −0.19, −0.18, −0.17, −0.16, −0.15, −0.14, −0.13, −0.12, −0.11, −0.10, −0.09, −0.08, −0.07, −0.06, −0.05, −0.04, −0.03, −0.02, −0.01, 0.00, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, and 0.20.

According to the “9103 Guideline for Hygroscopicity for Pharmaceuticals” in Part IV of the 2015 edition of the Chinese Pharmacopoeia, descriptions of hygroscopic characteristics and definitions of hygroscopic weight gain are as follows;
Deliquescent: Absorbs sufficient moisture to form a liquid;
Extremely hygroscopic: Hygroscopic weight gain is no less than 15%;
Hygroscopic: Hygroscopic weight gain is less than 15% but no less than 2%;
Slightly hygroscopic: Hygroscopic weight gain is less than 2% but no less than 0.2%;
Non-hygroscopic or nearly non-hygroscopic: Hygroscopic weight gain is less than 0.2%.

The term “differential scanning calorimetry or DSC” described in the present disclosure refers to measuring the temperature difference and heat flow difference between the sample and the reference during the process of heating or constant temperature of the sample, so as to characterize all physical changes and chemical changes related to thermal effects and obtain the phase change information of the sample.

The drying temperature described in the present disclosure generally ranges from 25° C. to 150° C., preferably from 40° C. to 80° C. Drying can be carried out under atmospheric pressure or under reduced pressure.

“Pharmaceutical composition” refers to a mixture containing one or more than one compound of formula (I) described herein or a pharmaceutically acceptable salt thereof and other chemical components, and other components, for example, a pharmaceutically acceptable carrier and an excipient. The pharmaceutical composition is intended to promote the administration to an organism, so as to facilitate the absorption of the active ingredient, thereby exerting biological activities.

The crystal forms in the present disclosure include, but are not limited to, solvates of the compound of formula (I). The solvents include, but are not limited to, water, methanol, ethanol, isopropanol, acetone, ethyl acetate, acetonitrile, isopropyl acetate, methyl tert-butyl ether, 2-butanone, tetrahydrofuran, dimethyl sulfoxide, N-methylpyrrolidone, methyl isobutyl ketone, dichloromethane, n-heptane, 1,4-dioxane, nitromethane, propylene glycol methyl ether, chloroform, N,N-dimethylformamide, N,N-dimethylacetamide, p-xylene, cyclohexane, dichloroethane, n-hexane, petroleum ether, n-octane, o-xylene, toluene, and isopropyl ether.

The term “solvate” described in the present disclosure includes, but is not limited to, a complex formed by combining the compound of formula (I) with a solvent.

### BRIEF DESCRIPTION OF THE DRAWINGS

FIG. 1 is the XRPD pattern of an amorphous form of the compound of formula (I).

FIG. 2 is the XRPD pattern of the crystal form A of the compound of formula (I).

FIG. 3 is the XRPD pattern of the crystal form B of the compound of formula (I).

FIG. 4 is the XRPD pattern of the crystal form C of the compound of formula (I).

FIG. 5 is the XRPD pattern of the crystal form D of the compound of formula (I).

FIG. 6 is the XRPD pattern of the crystal form E of the compound of formula (I).

FIG. 7 is the XRPD pattern of the crystal form F of the compound of formula (I).

FIG. 8 is the XRPD pattern of the crystal form G of the compound of formula (I).

FIG. 9 is the XRPD pattern of the crystal form H of the compound of formula (I).

FIG. 10 is the XRPD pattern of the crystal form I of the compound of formula (I).

FIG. 11 is the XRPD pattern of the crystal form J of the compound of formula (I).

FIG. 12 is the XRPD pattern of the crystal form K of the compound of formula (I).

FIG. 13 is the XRPD pattern of the crystal form L of the compound of formula (I).

FIG. 14 is the XRPD pattern of the crystal form M of the compound of formula (I).

FIG. 15 is the XRPD pattern of the crystal form N of the compound of formula (I).

FIG. 16 is the XRPD pattern of the crystal form O of the compound of formula (I).

FIG. 17 is the XRPD pattern of the crystal form P of the compound of formula (I).

FIG. 18 is the XRPD pattern of the crystal form Q of the compound of formula (I).

FIG. 19 is the XRPD pattern of the crystal form U of the compound of formula (I).

FIG. 20 is the XRPD pattern of the crystal form X of the compound of formula (I).

FIG. 21 is the XRPD pattern of the crystal form Y of the compound of formula (I).

FIG. 22 is the XRPD pattern of the crystal form V of the compound of formula (I).

FIG. 23 is the XRPD pattern of the crystal form R of the compound of formula (I).

FIG. 24 is the XRPD pattern of the crystal form S of the compound of formula (I).

FIG. 25 is the XRPD pattern of the crystal form T of the compound of formula (I).

FIG. 26: The efficacy data of the compound of Example 1 and the control example CC-92480 on NCI-H929 xenograft tumor in CB-17 SCID mice in vivo.

FIG. 27: Effects of the compound of Example 1 and the control example CC-92480 on the body weight of CB-17 SCID mice.

### DETAILED DESCRIPTION OF THE PREFERRED EMBODIMENT

The present disclosure will be explained in more detail below with reference to examples or experimental examples. The examples or experimental examples of the present disclosure are only used to illustrate the technical solutions of the present disclosure and do not limit the essence and scope of the present disclosure.

Experimental methods without specifying specific conditions in the examples of the present disclosure are generally in accordance with conventional conditions, or in accordance with the conditions recommended by the manufacturers of the raw materials or commercial products. Reagents without specifying a particular source are conventional reagents purchased from the market.

The structure of the compound was determined by nuclear magnetic resonance (NMR) spectroscopy and/or mass spectrometry (MS). NMR shift (S) is given in a unit of 10⁻⁶ (ppm). NMR spectra were determined using a Bruker AVANCE-400 nuclear magnetic resonance instrument, with deuterated dimethyl sulfoxide (DMSO-d₆), deuterated chloroform (CDCl₃), and deuterated methanol (CD₃OD) as determination solvents and tetramethylsilane (TMS) as an internal standard.

Mass spectra were determined using Agilent 1200/1290 DAD-6110/6120 Quadrupole MS liquid chromatography-mass spectrometry system (manufacturer: Agilent; MS model: 6110/6120 Quadrupole MS), Waters ACQuity UPLC-QD/SQD (manufacturer: Waters, MS model: Waters ACQuity Qda Detector/Waters SQ Detector), and THERMO Ultimate 3000-Q Exactive (manufacturer: THERMO, MS model: THERMO Q Exactive).

High performance liquid chromatography (HPLC) analysis was performed using Agilent HPLC 1200DAD, Agilent HPLC 1200VWD, and Waters HPLC e2695-2489 high performance liquid chromatographs.

Chiral HPLC analysis was performed on Agilent 1260 DAD high performance liquid chromatograph.

HPLC preparation was performed using Waters 2767, Waters 2767-SQ Detecor2, Shimadzu LC-20AP, and Gilson-281 preparative chromatographs.

Chiral preparation was performed on a Shimadzu LC-20AP preparative chromatograph.

A CombiFlash rapid preparation instrument used was Combiflash Rf200 (TELEDYNE ISCO).

Yantai Huanghai HSGF254 or Qingdao GF254 silica gel plates of specifications 0.15 mm to 0.2 mm were adopted for thin layer chromatography (TLC) analysis and 0.4 mm to 0.5 mm for TLC separation and purification.

Yantai Huanghai silica gel of 200 to 300-mesh was generally used as a carrier in silica gel column chromatography.

The mean inhibition of kinase and the IC₅₀ value were determined using a NovoStar microplate reader (BMG, Germany).

Known starting materials described herein may be synthesized using or according to methods known in the art, or may be purchased from ABCR GmbH & Co. KG, Acros Organics, Aldrich Chemical Company, Accela ChemBio Inc., Chembee Chemicals, and other companies.

In the examples, the reactions can be performed in an argon atmosphere or a nitrogen atmosphere unless otherwise specified.

The argon atmosphere or nitrogen atmosphere means that the reaction flask is connected to a balloon containing about 1 L of argon or nitrogen.

A hydrogen atmosphere means that the reaction flask is connected to a balloon containing about 1 L of hydrogen.

Parr 3916EKX hydrogenator, Qinglan QL-500 hydrogenator, or HC2-SS hydrogenator was used in the pressurized hydrogenation reactions.

The hydrogenation reactions usually involve 3 cycles of vacuumization and hydrogen purge.

A CEM Discover-S 908860 microwave reactor was used in the microwave reactions.

In the examples, a solution refers to an aqueous solution unless otherwise specified.

In the examples, the reaction temperature was room temperature, i.e., 20° C. to 30° C., unless otherwise specified.

The monitoring of the reaction progress in the examples was conducted by thin layer chromatography (TLC). The developing solvent for reactions, the eluent system for column chromatography purification and the developing solvent system for thin layer chromatography included: A: n-hexane/ethyl acetate system, and B: dichloromethane/methanol system. The volume ratio of the solvents was adjusted according to the polarity of the compound, or by adding a small amount of basic or acidic reagents such as triethylamine and acetic acid.

Test conditions for the instruments used in the experiments described in the present disclosure are as follows:

### 1. Differential Scanning Calorimeter (DSC)

Instrument model: Mettler Toledo DSC 3+ STARe System
Purge gas: Nitrogen; Nitrogen flow rate: 50 mL/min
Heating rate: 10.0° C./min
Temperature range: 25 to 350° C.

### 2. X-Ray Powder Diffraction (XRPD)

Instrument model: BRUKER D8 Discover X-ray Powder Diffractometer
Radiation: Monochromatic Cu-Kα radiation (λ=1.5418 Å)
Scanning method: θ/2θ, Scanning range (20 range): 3 to 50°
Voltage: 40 kV, Current: 40 mA

### 3. Thermogravimetric Analysis (TGA)

Instrument model: Mettler Toledo TGA2
Purge gas: Nitrogen; Nitrogen flow rate: 50 mL/min
Heating rate: 10.0° C./min
Temperature range: 25 to 350° C.

### Example 1: Preparation of Compound of Formula (I)

### (S)-4-(4-((5-(((2-(2,6-dioxopiperidin-3-yl)-1-oxoisoindolin-4-yl)oxy)methyl)pyridin-2-yl)thio)piperidin-1-yl)-3-fluorobenzonitrile (Compound 1)

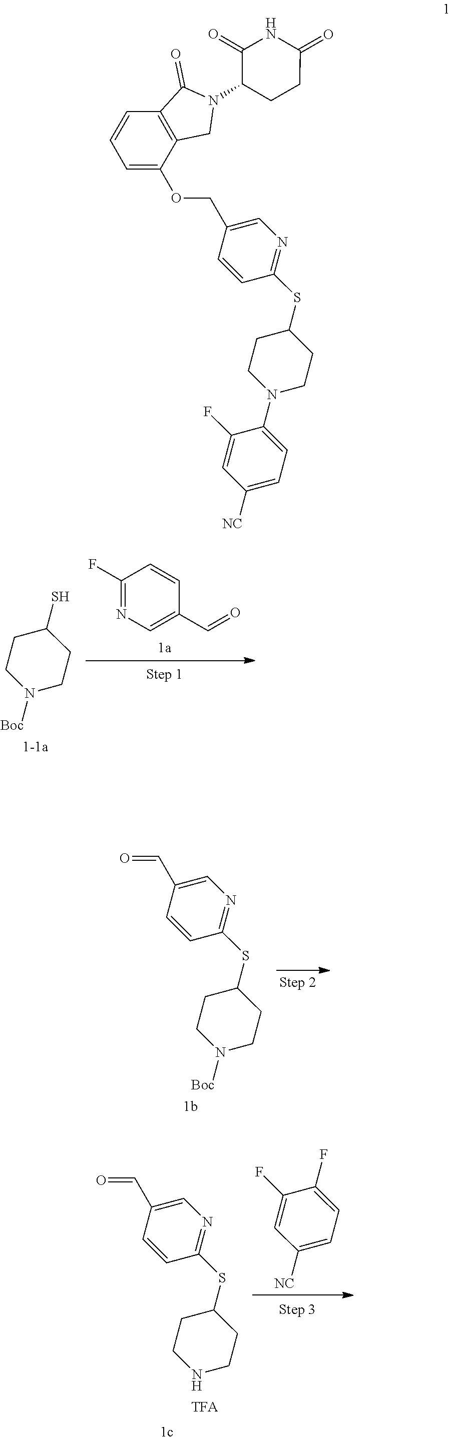 [SMILES: N#Cc1ccc(N2CCC(Sc3ccc(COc4cccc5c4CN([C@H]4CCC(=O)NC4=O)C5=O)cn3)CC2)c(F)c1 ; CC(C)(C)OC(=O)N1CCC(S)CC1 ; O=Cc1ccc(F)nc1 ; CC(C)(C)OC(=O)N1CCC(Sc2ccc(C=O)cn2)CC1 ; O=Cc1ccc(SC2CCNCC2)nc1 ; N#Cc1ccc(F)c(F)c1]

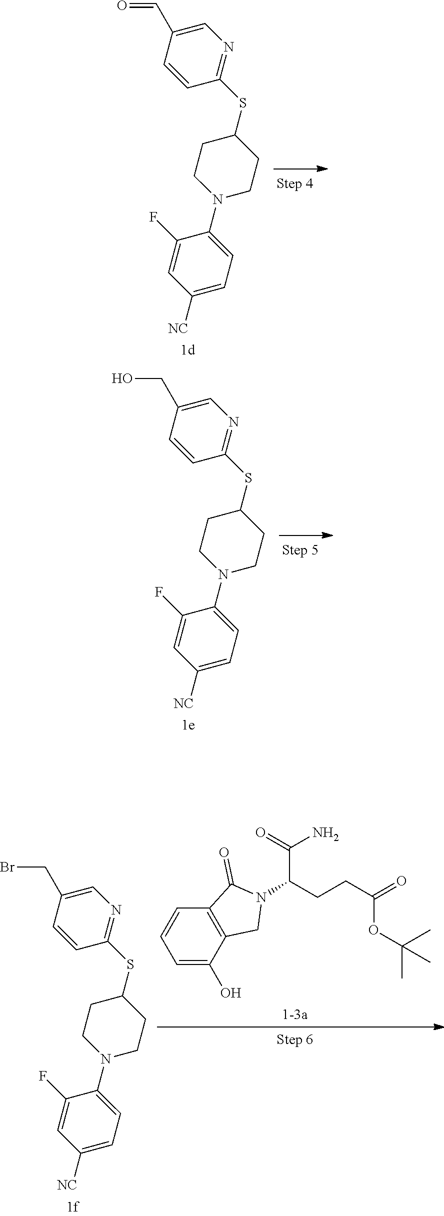

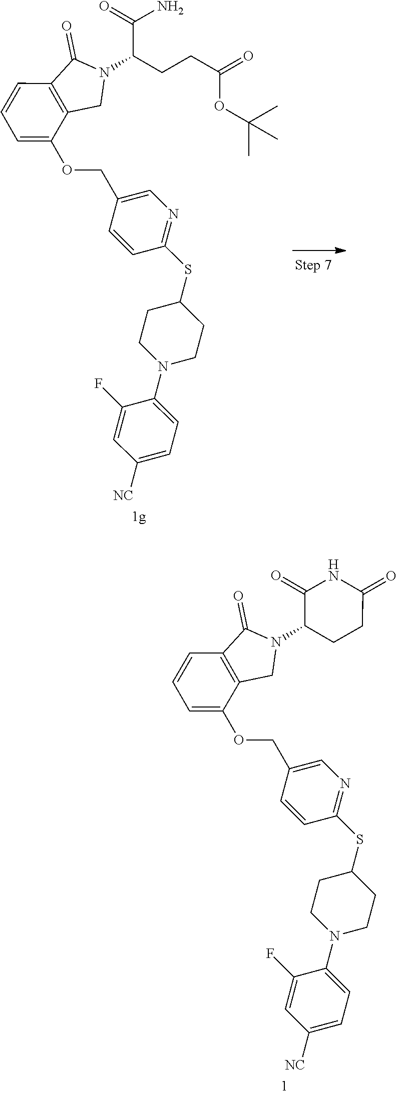 [SMILES: CC(C)(C)OC(=O)CC[C@@H](C(N)=O)N1Cc2c(OCc3ccc(SC4CCN(c5ccc(C#N)cc5F)CC4)nc3)cccc2C1=O ; N#Cc1ccc(N2CCC(Sc3ccc(COc4cccc5c4CN([C@H]4CCC(=O)NC4=O)C5=O)cn3)CC2)c(F)c1]

### Step 1

### tert-Butyl 4-((5-formylpyridin-2-yl)thio)piperidine-1-carboxylate 1b

Compound 1-1a (700 mg, 3.22 mmol), 6-fluoropyridine-3-carboxaldehyde 1a (443 mg, 3.54 mmol), and potassium carbonate (1.11 g, 8.05 mmol) were added to N,N-dimethylformamide (10 mL). The reaction mixture was heated to 80° C. and reacted for 1 hour. The reaction mixture was diluted with water (30 mL) and extracted with ethyl acetate (40 mL×3). The organic phases were combined, washed with a saturated sodium chloride solution (100 mL), dried over anhydrous sodium sulfate, and filtered, and the filtrate was concentrated under reduced pressure. The residue was purified by column chromatography with an eluent system B to give the title compound 1b (1.0 g, yield: 96%).

MS m/z (ESI): 267.1 [M−55].

### Step 2

### 6-(Piperidin-4-ylthio)nicotinaldehyde trifluoroacetate 1c

Compound 1b (950 mg, 2.95 mmol) was dissolved in dichloromethane (10 mL), and trifluoroacetic acid (2 mL) was slowly added thereto under an ice bath. The reaction mixture was reacted for 1 hour. The reaction mixture was concentrated and dried to give the title compound 1c (, and the crude product was directly used in the next step without purification.

MS m/z (ESI): 223.1 [M+1].

### Step 3

### 3-Fluoro-4-(4-((5-formylpyridin-2-yl)thio)piperidin-1-yl)benzonitrile 1d

Compound 1c (760 mg, 2.94 mmol), 3,4-difluorobenzonitrile (817 mg, 5.87 mmol), and potassium carbonate (1.22 g, 8.81 mmol) were added to N,N-dimethylformamide (15 mL), and the reaction mixture was heated to 80° C. and reacted overnight. The reaction mixture was added with water (50 mL) and extracted with ethyl acetate (50 mL×3). The organic phases were combined, washed with a saturated sodium chloride solution (50 mL×3), dried over anhydrous sodium sulfate, and filtered, and the filtrate was concentrated under reduced pressure. The resulting residue was purified by column chromatography with an eluent system B to give the title compound 1d (850 mg, yield: 84%).

MS m/z (ESI): 342.1 [M+1].

### Step 4

### 3-Fluoro-4-(4-((5-(hydroxymethyl)pyridin-2-yl)thio)piperidin-1-yl)benzonitrile 1e

Compound 1d (600 mg, 1.76 mmol) was added to methanol (10 mL) under an ice bath, followed by the slow addition of sodium borohydride (133 mg, 3.51 mmol), and the reaction mixture was reacted for 1 hour. The reaction was quenched with water (10 mL), and the reaction mixture was extracted with ethyl acetate (30 mL×3). The organic phases were combined, washed with a saturated sodium chloride solution (30 mL), dried over anhydrous sodium sulfate, and filtered, and the filtrate was concentrated under reduced pressure. The residue was purified by column chromatography with an eluent system B to give the title compound 1e (580 mg, yield: 96%).

MS m/z (ESI): 344.1 [M+1].

### Step 5

### 4-(4-((5-(Bromomethyl)pyridin-2-yl)thio)piperidin-1-yl)-3-fluorobenzonitrile if

Compound 1e (200 mg, 0.582 mmol) was added to dichloromethane (6 mL), followed by the sequential addition of triphenylphosphine (199 mg, 0.757 mmol) and carbon tetrabromide (251 mg, 0.757 mmol), and the reaction mixture was reacted for 2 hours. The reaction mixture was concentrated. The residue was purified by column chromatography with an eluent system B to give the title compound if (190 mg, yield: 80%).

MS m/z (ESI): 406.0 [M+1]; 408.0 [M+3].

### Step 6

### tert-Butyl (S)-5-amino-4-(4-((6-((1-(4-cyano-2-fluorophenyl)piperidin-4-yl)thio)pyridin-3-yl)methoxy)-1-oxoisoindolin-2-yl)-5-oxopentanoate 1g

tert-Butyl (S)-5-amino-4-(4-hydroxy-1-oxoisoindolin-2-yl)-5-oxopentanoate 1-3a (82 mg, 0.246 mmol, prepared using the well-known method described in “Journal of Medicinal Chemistry”, 2020, 63 (13), 6648-6676) and anhydrous potassium carbonate (65 mg, 0.468 mmol) were added to N,N-dimethylformamide (3 mL), followed by the addition of compound if (95 mg, 0.244 mmol), and the reaction mixture was reacted for 2 hours. The reaction mixture was poured into ice water (10 mL), and the resulting mixture was extracted with ethyl acetate (30 mL×3). The organic phases were combined, washed with a saturated sodium chloride solution (20 mL×2), dried over anhydrous sodium sulfate, and filtered, and the filtrate was concentrated under reduced pressure. The residue was purified by column chromatography with an eluent system B to give the title compound 1g (145 mg, yield: 94%).

MS m/z (ESI): 660.2 [M+1].

### Step 7

### (S)-4-(4-((5-(((2-(2,6-Dioxopiperidin-3-yl)-1-oxoisoindolin-4-yl)oxy)methyl)pyridin-2-yl)thio)piperidin-1-yl)-3-fluorobenzonitrile 1

Compound 1g (60 mg, 0.091 mmol) was added to acetonitrile (5 mL), followed by the addition of benzenesulfonic acid (16 mg, 0.091 mmol) at room temperature, and the reaction mixture was reacted at 80° C. for 8 hours. The reaction mixture was concentrated under reduced pressure to remove the solvent. The resulting residue was subjected to high performance liquid chromatography (Gilson GX-281, elution system: 10 mmol/L aqueous ammonium bicarbonate solution and acetonitrile, gradient of acetonitrile: 60%-80%, flow rate: 30 mL/min) to give the title compound 1 (42 mg, yield: 78%).

Upon detection using an X-ray powder diffractometer, the title compound 1 was detected as amorphous.

MS m/z (ESI): 586.4 [M+1].

¹H NMR (500 MHz, DMSO-d6): δ 10.98 (s, 1H), 8.60 (s, 1H), 7.78 (dd, 1H), 7.69 (dd, 1H), 7.59-7.48 (m, 2H), 7.38-7.33 (m, 3H), 7.16 (t, 1H), 5.24 (s, 2H), 5.12 (dd, 1H), 4.42 (d, 1H), 4.26 (d, 1H), 4.07-3.99 (m, 1H), 3.56-3.48 (m, 2H), 3.13-3.03 (m, 2H), 2.96-2.87 (m, 1H), 2.63-2.55 (m, 1H), 2.46-2.38 (m, 1H), 2.21-2.12 (m, 2H), 2.02-1.94 (m, 1H), 1.82-1.70 (m, 2H).

### Biological Evaluation

### Test Example 1. Biological Evaluation of NCI-H929 Proliferation Experiment

The following method was used to determine the inhibitory activity of the compounds of the present disclosure on the proliferation of NCI-H929 cells. The experimental method was briefly described as follows.

NCI-H929 cells (ATCC, CRL-9068) were cultured in a complete medium (RPMI 1640 medium (Hyclone, SH30809.01) containing 10% fetal bovine serum (Corning, 35-076-CV) and 0.05 mM 2-mercaptoethanol (Sigma, M3148)). On the first day of the experiment, NCI-H929 cells were seeded in a 96-well plate at a density of 6000 cells/well with a complete medium to form 100 μL of cell suspension per well, and 10 μL of test compound prepared in a complete medium and diluted in a gradient was added to each well. The compound was firstly dissolved in DMSO, with an initial concentration of 10 mM, and then subjected to serial dilution at a 5-fold concentration gradient for a total of 9 concentration points, with a blank control of 100% DMSO. Another 5 μL of the compound dissolved in DMSO was added to 95 μL of complete medium, i.e., the compound was diluted 20-fold with the complete medium. Finally the compound diluted in the complete medium at 10 μL/well was added to the cell suspension, wherein the final concentrations of the compound were 9 concentration points obtained by 5-fold gradient dilution starting from 50 μM. A blank control containing 0.5% DMSO was set. The plate was incubated in a cell incubator at 37° C. with 5% CO₂ for 5 days. On the sixth day, the 96-well cell culture plate was taken out, added with a CellTiter-Glo® luminescent cell activity detect reagent (Promega, G7573) at 50 μL/well, left to stand at room temperature for 10 min, and read for the luminescence signal values using a multi-mode microplate reader (PerkinElmer, EnVision 2015). IC₅₀ values for the inhibitory activity of the compounds were calculated using Graphpad Prism software, with the results shown in Table I.

TABLE I
IC₅₀ values for the inhibition of proliferation of NCI-
H929 cells by the compounds of the present disclosure

| Compound | IC₅₀ (nM) |
|---|---|
| 1 | 0.02 |

Conclusion: Compound 1 of the present disclosure has good inhibitory activity for the proliferation of NCI-H929 cells.

### Test Example 2. Pharmacodynamic Test

### 1. Objective

This experiment was performed to evaluate the inhibition effect of the compound of Example 1 and the control example CC-92480 on the growth of human multiple myeloma cell NCI-H929 xenograft tumor in CB-17 SCID mice.

### 2. Test Compounds

### Compound of Example 1

control example CC-92480 (see compound 2 of WO2019014100A1, synthesized according to the method disclosed therein)

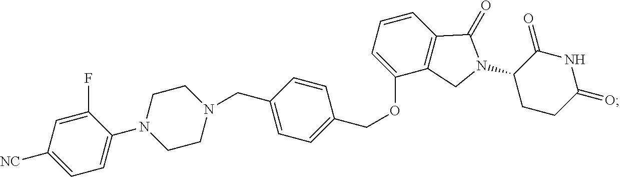 [SMILES: N#Cc1ccc(N2CCN(Cc3ccc(COc4cccc5c4CN([C@H]4CCC(=O)NC4=O)C5=O)cc3)CC2)c(F)c1]

Solutions of the compound of Example 1 and the control example CC-92480 were prepared with 5% DMSO+20% PEG400+70% (10% TPGS)+5% (1% HPMC K100LV).

### 3. Experimental Procedures and Materials

### 3.1 Experimental Animals and Housing Conditions

Thirty CB-17 SCID female mice, purchased from Beijing Vital River Laboratory Animal Technology Co., Ltd. (certificate No. 20170011006049, SCXK (Shanghai) 2017-0011), weighing about 19 g at the time of purchase, were bred at 5 mice/cage (in a 12/12 hour light/dark cycle, at a constant temperature of 23±1° C. and humidity of 50%-60%) and had free access to food and water.

### 3.2 Group of Animals:

After adaptive feeding, the CB-17 SCID mice were grouped as follows:

| Group | n | Route of administration |
|---|---|---|
| Vehicle control | 7 | 5% DMSO + 20% PEG400 + 70% (10% |
| | | TPGS) + 5% (1% HPMC K100LV) (i.g/qd) |
| Example 1 | 7 | 1 mg/kg (i.g/qd) |
| CC-92480 | 7 | 1 mg/kg (i.g/qd) |

Note:
qd denotes once a day; i.g. denotes intragastric administration.

### 3.3. Procedures:

NCI-H929 cells in a logarithmic growth phase were inoculated subcutaneously into the right flank of 30 female CB-17 SCID mice at 5×10⁶ cells/mouse/100 μL (containing 50 L of Matrigel). After 10 days, when the tumor volume of the tumor-bearing mice reached about 200 mm³, the mice were randomly divided into 3 groups according to the tumor volume and body weight: vehicle control group, CC-92480-1 mpk, compound of Example 1-1 mpk, with 7 mice in each group. The day of grouping was set as Day 0 (DO), and intragastric administration was performed once a day for 11 days (Table 2). For the tumor-bearing mice, the tumor volume was measured with a vernier caliper and the body weight was measured with a balance twice a week, and the data were recorded. The tumor-bearing animals were euthanized as the experimental endpoint when the tumor volume reached 2000 mm³ or when most tumors showed rupture or when the tumor-bearing animals showed 20% of body weight loss.

### 3.4 Data Statistics

All data were plotted and statistically analyzed using Excel and GraphPad Prism 5 software.

The tumor volume (V) was calculated as follows: V=1/2×a×b², where a and b represent length and width, respectively.

The relative tumor proliferation rate T/C (%)=(T−T₀)/(C−C₀)×100(%), where T and C are the tumor volume of animals at the end of the experiment in the treatment group and control group, respectively; T₀ and C₀ are the tumor volume of animals at the beginning of the experiment in the treatment group and control group, respectively.

Tumor growth inhibition TGI (%)=1−T/C (%), and when TGI (%) exceeds 100%, no specific value will be shown, and it is expressed only by >100%.

Tumor regression (%)=[(T₀−T)/T₀]×100(%).

### 4. Results

The data on the efficacy of the compound of Example 1 and the control example CC-92480 on NCI-H929 xenograft tumor in CB-17 SCID mice are shown in Table II below and FIG. 26.

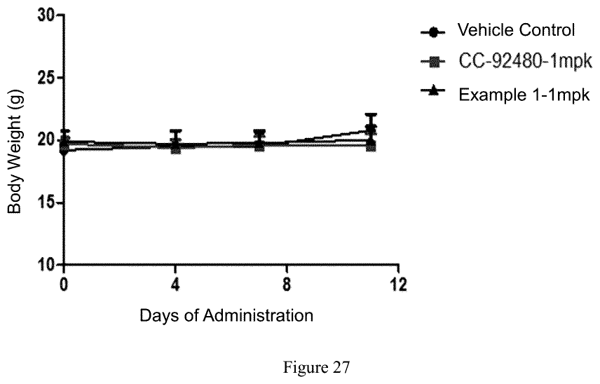

The effect of the compound of Example 1 and the control example CC-92480 on the body weight of CB-17 SCID mice is shown in FIG. 27.

TABLE II
Efficacy of the compound of the present disclosure
on NCI-H929 xenograft tumor in CB-17 SCID mice

| | | | | | | | | p | Number of |
|---|---|---|---|---|---|---|---|---|---|
| | | | | | | | % Tumor | (v.s. | remaining |
| | Route of | | Mean tumor volume (mm³) | | | | regression | Vehicle | animals/ |
| Group | administration | | D 0 | SEM | D 11 | SEM | D 11 | control) | group |
| Vehicle | qd/11 d | po | 156.3 | 15.9 | 1711.7 | 190.1 | / | / | 7/7 |
| control | | | | | | | | | |
| Example 1 | qd/11 d | po | 155.4 | 16.5 | 18.6 | 1.8 | 88 | <0.001 | 7/7 |
| CC-92480 | qd/11 d | po | 157.2 | 13.6 | 103.6 | 27.1 | 34 | <0.001 | 7/7 |

Note:
qd denotes once a day; po denotes oral administration.

### 5. Conclusion

The compound of Example 1 was administered once a day starting from 10 days after tumor cell transplantation, and significant tumor volume regressions occurred 11 days after administration. Upon calculation, the tumor inhibition rate is >100%, and the tumor regression rate is 88%. The statistical difference (p<0.05) is obtained when compared with an equal dose of CC-92480 at the end point of the experiment, and the administration has no influence on the body weight of mice. Under the same conditions, the tumor regression rate of the control example CC-92480 is 34%.

### Test Example 3. Pharmacokinetic Evaluation

### 1. Overview

The drug concentration in the plasma of the test animals (mice) at different time points after intragastric administration of the compound of Example 1 and the control example CC-92480 was determined by an LC/MS/MS method. The pharmacokinetic behavior of the compound of the present disclosure in mice was studied and its pharmacokinetic profile was evaluated.

### 2. Test Scheme

### 2.1. Test Compounds

Compound of Example 1 and control example CC-92480.

### 2.2. Test Animals

Eighteen mice, female, purchased from Beijing Vital River Laboratory Animal Technology Co., Ltd., with animal production license number of SCXK (Shanghai) 2017-0005.

### 2.3. Drug Preparation

A certain amount of compound of Example 1 was weighed, dissolved by adding 5% by volume of DMSO and 5% Tween 80 (Shanghai Titan Scientific Co., Ltd.), and then prepared into a 0.1 mg/mL clear solution by adding 90% normal saline.

A certain amount of the control example CC-92480 was weighed, dissolved by adding 5% by volume of DMSO and 5% Tween 80 (Shanghai Titan Scientific Co., Ltd.), and then prepared into a 0.1 mg/mL clear solution by adding 90% normal saline.

### 2.4. Administration

Nine mice were intragastrically administered with the compound of Example 1 at a dose of 2 mg/kg and at a volume of 0.2 mL/10 g.

Nine mice were intragastrically administered with the control example CC-92480 at a dose of 2 mg/kg and at a volume of 0.2 mL/10 g.

### 3. Procedures

Mice were intragastrically administered with the compound of Example 1 and the control example CC-92480, and 0.2 mL of blood was collected before the administration and 0.25 h, 0.5 h, 1.0 h, 2.0 h, 4.0 h, 6.0 h, 8.0 h, 11.0 h and 24.0 h after the administration (3 animals at each time point), placed in EDTA-K2 anticoagulation tubes, and centrifuged at 10,000 rpm for 1 min (4° C.). Plasma was separated within 1 hour, and stored at −20° C. for testing. The process from blood sampling to centrifugation was performed under an ice bath.

The content of the test compounds at different concentrations in mouse plasma after the administration was determined: 25 μL of mouse plasma at each time point after the administration was mixed with 50 μL (100 ng/mL) of internal standard solution camptothecin (National Institutes for Drug Control) and 175 μL of acetonitrile. The mixture was vortexed for 5 min and centrifuged for 10 min (3700 rpm), and 1 μL of the supernatant of the plasma sample was taken for LC/MS/MS assay (API4000 triple quadrupole tandem mass spectrometer, Applied Biosystems, USA; Shimadzu, LC-30AD ultra high performance liquid chromatography system, Shimadzu, Japan).

### 4. Pharmacokinetic Parameter Results

Pharmacokinetic parameters for the compound of the present disclosure are shown in Table III below.

TABLE III
Pharmacokinetic parameters of the compound of the present disclosure

| | Pharmacokinetic experiment (2 mg/kg) | | | | | |
|---|---|---|---|---|---|---|
| | | | | | | Apparent |
| | Plasma | Area under | Half- | Residence | Clearance | volume of |
| | concentration | curve | life | time | rate | distribution |
| | Cmax | AUC | T1/2 | MRT | CL/F | Vz/F |
| No. | (ng/mL) | (ng /mL * h) | (h) | (h) | (mL/min/kg) | (ml/kg) |
| Example 1 | 799 | 4686 | 2.9 | 4.6 | 7.1 | 1785 |
| CC-92480 | 692 | 1937 | 1.3 | 2.3 | 17.2 | 1961 |

Conclusion: The compound of the present disclosure demonstrates good absorption profile and has significant pharmacokinetic advantages.

### Test Example 4. Evaluation of Plasma Stability of the Compound of the Present Disclosure

### 1. Abstract

The stability of the compound of Example 1 and the control example CC-92480 after incubation in cryopreserved monkey plasma at 37° C. for 0 min, 15 min, 30 min, 60 min, 120 min, 180 min and 240 min was determined quantitatively by LC-MS/MS.

### 2. Test Protocol

### 2.1. Test Compounds

Compound of Example 1 and control example CC-92480.

### 2.2 Test Plasma

Monkey plasma was purchased from Shanghai Medicilon Inc.

### 2.3 Preparation of Compound Solutions

A certain amount of the compound of Example 1 was weighed and prepared with DMSO to obtain a 30 mM stock solution. A certain volume of the stock solution was diluted into a solution I at a concentration of 1600 μM with DMSO; and a certain volume of the solution I at 1600 μM was diluted with 50% methanol to obtain a working solution II at a concentration of 16 μM. A30 mM stock solution, a 1600 μM solution I′, and a 16 μM working solution II′ of CC-92480 were prepared as described above.

### 2.4 Sample Incubation

5 μL of the working solutions at 16 μM of the compound of Example 1 and the control example CC-92480 were each added to 75 μL of plasma to make the final concentration of the compounds of 1 μM. The samples were incubated under a 37° C. water bath for 0 min, 15 min, 30 min, 60 min, 90 min, 120 min, and 180 min. After the incubation, 240 μL of acetonitrile containing the internal standard was added, and the plate was shaken on a shaker at 800 rpm for 10 min and centrifuged on a centrifugation at 4° C. at 3700 rpm for 20 min. The supernatant was analyzed by LC-MS with a sample injection volume of 2 μL.

### 3. Results

The conversion of the compound of the present disclosure in monkey plasma is shown in Table IV below.

TABLE IV
Stability data of the compound of the
present disclosure in monkey plasma

| No. | T_{1/2} (min)/monkey |
|---|---|
| Example 1 | 806 |
| CC-92480 | 199 |

Conclusion: the compound of the present disclosure has stability advantages in monkey plasma.

### Example 2: Preparation of Amorphous Form of Compound of Formula (I)

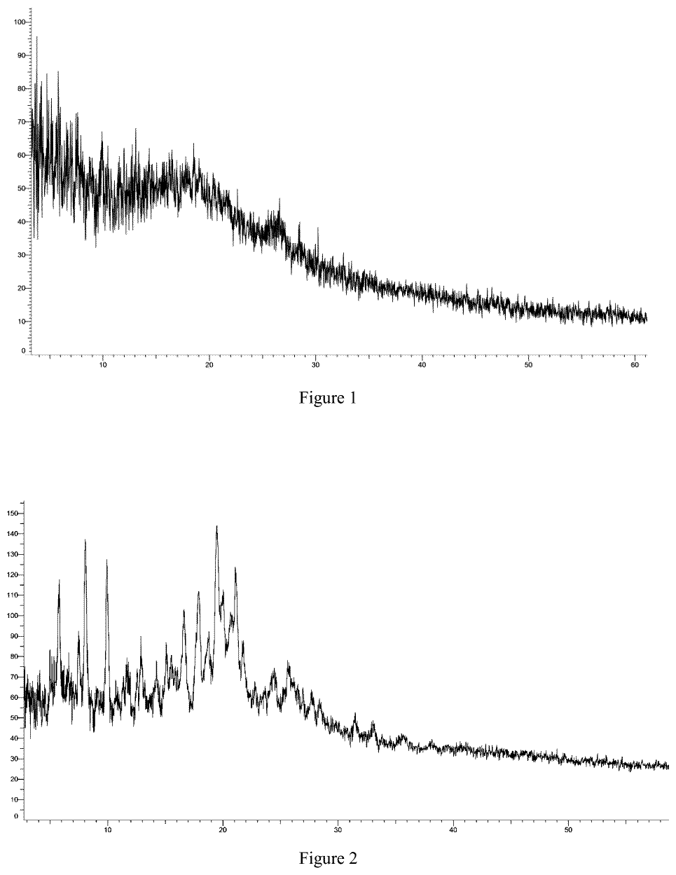

Compound of formula (I) (10 mg) was added to propylene glycol methyl ether (1.0 mL), slurried at room temperature, and centrifuged. The solid was dried to obtain the product. The product was detected as amorphous form by an X-ray powder diffractometer and its XRPD pattern is shown in FIG. 1.

### Example 3: Preparation of Crystal Form a of Compound of Formula (I)

Compound of formula (I) (7.3 g) was added to dichloromethane (300 mL), dissolved with methanol (30 mL), and water (300 mL) was added thereto. The reaction mixture was subjected to rotary evaporation to remove the majority of the dichloromethane and to precipitate a large amount of solid, filtered and the solid was obtained and then dried overnight. The solid was dissolved in dichloromethane (500 mL), filtered under reduced pressure to obtain the filtrate (500 mL), which was dried over anhydrous sodium sulfate, filtered, and ethyl acetate (200 mL) was added. The mixture was dried under rotary evaporation to obtain the product. After X-ray powder diffraction detection, the product was defined as crystal form A. The XRPD pattern is shown in FIG. 2, and its characteristic peak positions are shown in Table 1. DSC pattern showed endothermic peaks at 155.26° C., 183.19° C., and 244.74° C. TGA pattern showed a weight loss of 9.48% from 25° C. to 220° C.

TABLE 1

| Peak No. | 2θ value [° or degree] | d[Å] | Relative intensity % |
|---|---|---|---|
| 1 | 5.765 | 15.31661 | 67.0 |
| 2 | 7.465 | 11.83234 | 40.7 |
| 3 | 8.061 | 10.95880 | 100.0 |
| 4 | 9.925 | 8.90513 | 80.2 |
| 5 | 11.674 | 7.57437 | 30.1 |
| 6 | 12.890 | 6.86264 | 36.5 |
| 7 | 14.270 | 6.20150 | 17.7 |
| 8 | 15.085 | 5.86862 | 31.7 |
| 9 | 16.632 | 5.32575 | 48.4 |
| 10 | 17.900 | 4.95150 | 56.1 |
| 11 | 18.715 | 4.73745 | 23.5 |
| 12 | 19.469 | 4.55574 | 88.1 |
| 13 | 19.860 | 4.46700 | 43.8 |
| 14 | 20.696 | 4.28837 | 36.6 |
| 15 | 21.115 | 4.20425 | 58.4 |
| 16 | 21.708 | 4.09064 | 22.9 |
| 17 | 24.393 | 3.64618 | 12.3 |
| 18 | 25.669 | 3.46771 | 14.1 |

### Example 4: Preparation of Crystal Form a of Compound of Formula (I)

Compound of formula (I) (10 mg) was added to water (1 mL), slurried at room temperature, and centrifuged. The solid was dried under vacuum to obtain the product. After X-ray powder diffraction detection, the product was defined as crystal form A.

### Example 5: Preparation of Crystal Form a of Compound of Formula (I)

Compound of formula (I) (10 mg) was added to acetone (1 mL), slurried, and centrifuged. The solid was dried under vacuum to obtain the product. After X-ray powder diffraction detection, the product was defined as crystal form A.

### Example 6: Preparation of Crystal Form a of Compound of Formula (I)

Compound of formula (I) (10 mg) was added to THE (300 uL), dissolved to clarification, then n-heptane (450 uL) was added thereto to precipitate a solid. The mixture was slurried at room temperature and centrifuged. The solid was dried under vacuum to obtain the product. After X-ray powder diffraction detection, the product was defined as crystal form A.

### Example 7: Preparation of Crystal Form B of Compound of Formula (I)

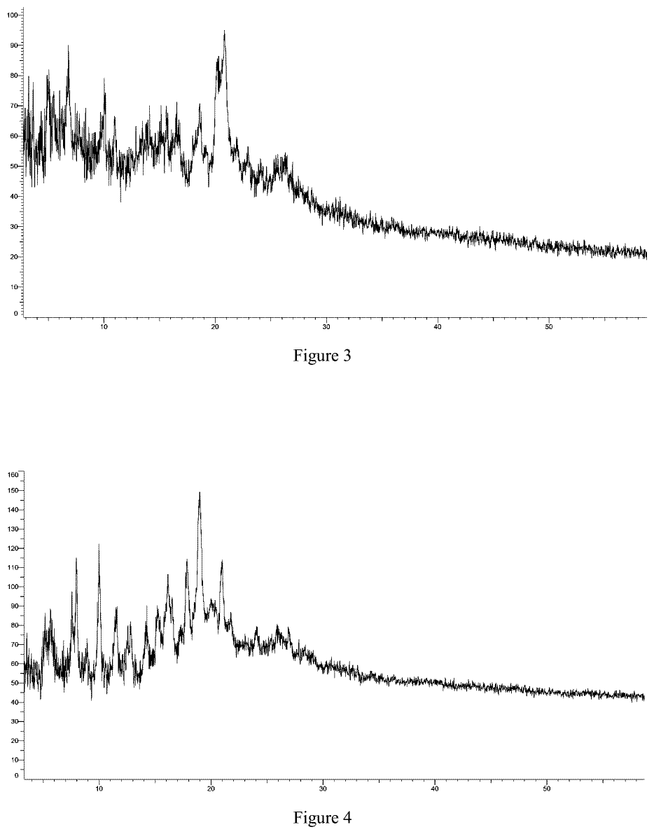

Compound of formula (I) (150 mg) was added to acetonitrile/methanol (1.0 mL, V/V=1:1), slurried, and centrifuged. The solid was dried under vacuum to obtain the product. After X-ray powder diffraction detection, the product was defined as crystal form B. The XRPD pattern is shown in FIG. 3, and its characteristic peak positions are shown in Table 2. DSC pattern showed endothermic peaks at 182.74° C. and 245.37° C. TGA pattern showed a weight loss of 4.57% from 25° C. to 225° C.

TABLE 2

| Peak No. | 2θ value [° or degree] | d[Å] | Relative intensity (%) |
|---|---|---|---|
| 1 | 4.977 | 17.74069 | 51.6 |
| 2 | 6.788 | 13.01194 | 59.7 |
| 3 | 10.047 | 8.79717 | 59.1 |
| 4 | 10.972 | 8.05760 | 47.9 |
| 5 | 14.143 | 6.25730 | 9.8 |
| 6 | 15.684 | 5.64563 | 23.7 |
| 7 | 18.547 | 4.78018 | 37.6 |
| 8 | 20.088 | 4.41673 | 65.3 |
| 9 | 20.840 | 4.25906 | 100.0 |
| 10 | 21.982 | 4.04031 | 10.9 |
| 11 | 22.907 | 3.87923 | 5.8 |
| 12 | 24.096 | 3.69042 | 4.8 |
| 13 | 25.505 | 3.48961 | 2.5 |

### Example 8: Preparation of Crystal Form C of Compound of Formula (I)

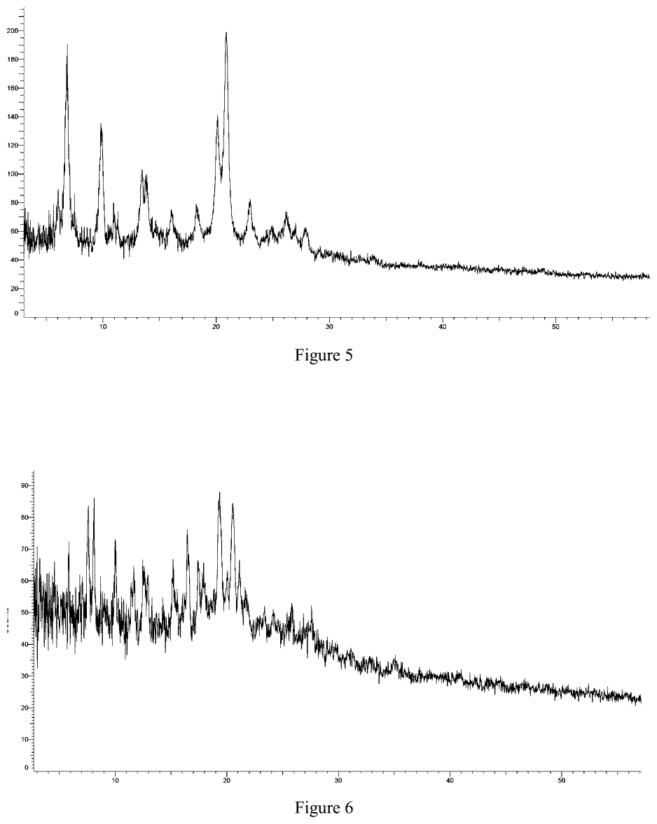

Compound of formula (I) (10 mg) was added to ethanol (1.0 mL), slurried at room temperature, and centrifuged. The solid was dried under vacuum to obtain the product. After X-ray powder diffraction detection, the product was defined as crystal form C. The XRPD pattern is shown in FIG. 4, and its characteristic peak positions are shown in Table 3. DSC pattern showed endothermic peaks at 153.48° C., 182.76° C., and 244.05° C. TGA pattern showed a weight loss of 3.00% from 25° C. to 215° C.

TABLE 3

| Peak No. | 2θ value [° or degree] | d[Å] | Relative intensity (%) |
|---|---|---|---|
| 1 | 3.533 | 24.98692 | 29.6 |
| 2 | 5.653 | 15.62173 | 51.4 |
| 3 | 7.608 | 11.61088 | 44.3 |
| 4 | 7.974 | 11.07924 | 81.3 |
| 5 | 8.790 | 10.05224 | 20.9 |
| 6 | 9.989 | 8.84772 | 89. |
| 7 | 11.505 | 7.68553 | 45.7 |
| 8 | 12.493 | 7.07950 | 18.7 |
| 9 | 12.798 | 6.91147 | 37.1 |
| 10 | 14.265 | 6.20385 | 38.7 |
| 11 | 15.277 | 5.79513 | 32.5 |
| 12 | 16.143 | 5.48617 | 54.5 |
| 13 | 17.860 | 4.96247 | 57.2 |
| 14 | 18.992 | 4.66919 | 100.0 |
| 15 | 19.943 | 4.44843 | 17.1 |
| 16 | 20.972 | 4.23258 | 50.1 |
| 17 | 21.773 | 4.07853 | 19.0 |
| 18 | 23.995 | 3.70564 | 8.9 |
| 19 | 25.912 | 3.43567 | 12.1 |
| 20 | 26.958 | 3.30473 | 8.0 |

### Example 9: Preparation of Crystal Form C of Compound of Formula (I)

Compound of formula (I) (10 mg) was added to 10% water/acetone (0.9 mL). The mixture was volatilized and crystallized to obtain the product. After X-ray powder diffraction detection, the product was defined as crystal form C.

### Example 10: Preparation of Crystal Form D of Compound of Formula (I)

Compound of formula (I) (10 mg) was added to acetonitrile (1.0 mL), slurried, and centrifuged. The solid was dried at 30° C. under vacuum to obtain the product. After X-ray powder diffraction detection, the product was defined as crystal form D. The XRPD pattern is shown in FIG. 5, and its characteristic peak positions are shown in Table 4. DSC pattern showed endothermic peaks at 156.12° C. and 245.38° C. TGA pattern showed a weight loss of 1.17% from 25° C. to 205° C.

TABLE 4

| Peak No. | 2θ value [° or degree] | d[Å] | Relative intensity (%) |
|---|---|---|---|
| 1 | 6.831 | 12.92916 | 88.1 |
| 2 | 9.845 | 8.97708 | 59.0 |
| 3 | 10.927 | 8.09061 | 14.6 |
| 4 | 13.453 | 6.57630 | 35.9 |
| 5 | 13.861 | 6.38376 | 32.7 |
| 6 | 16.096 | 5.50187 | 13.2 |
| 7 | 18.225 | 4.86376 | 14.8 |
| 8 | 20.117 | 4.41049 | 56.5 |
| 9 | 20.891 | 4.24885 | 100.0 |
| 10 | 23.006 | 3.86268 | 18.4 |
| 11 | 24.829 | 3.58314 | 2.0 |
| 12 | 26.132 | 3.40731 | 11.4 |
| 13 | 27.001 | 3.29960 | 3.5 |
| 14 | 27.913 | 3.19380 | 6.2 |

### Example 11: Preparation of Crystal Form E of Compound of Formula (I)

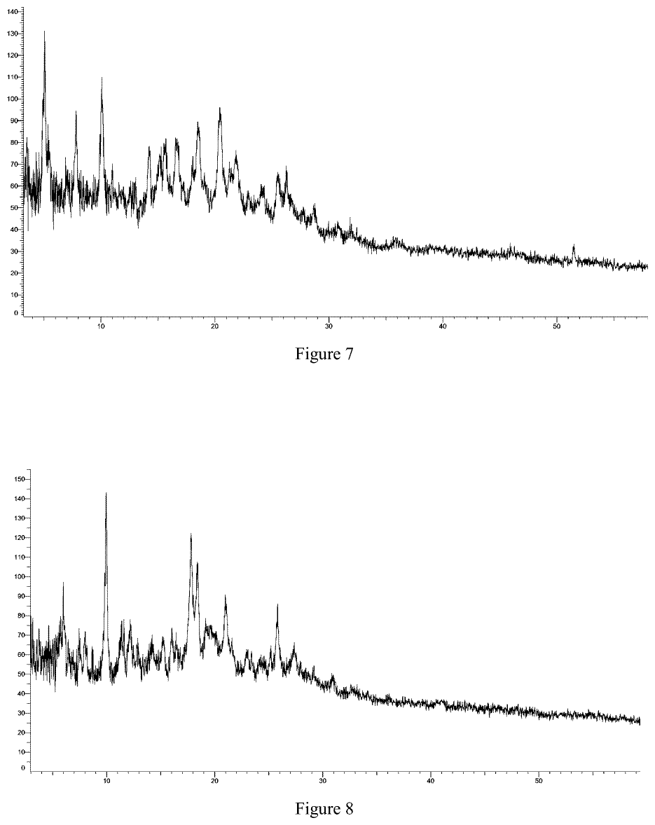

Compound of formula (I) (10 mg) was added to 10% water/methanol (1.0 mL), slurried, and centrifuged. The solid was dried under vacuum to obtain the product. After X-ray powder diffraction detection, the product was defined as crystal form E. The XRPD pattern is shown in FIG. 6, and its characteristic peak positions are shown in Table 5. DSC pattern showed endothermic peaks at 182.71° C. and 245.00° C. TGA pattern showed a weight loss of 0.68% from 25° C. to 235° C.

TABLE 5

| Peak No. | 2θ value [° or degree] | d[Å] | Relative intensity (%) |
|---|---|---|---|
| 1 | 5.864 | 15.05957 | 59.3 |
| 2 | 7.573 | 11.66418 | 100.0 |
| 3 | 8.087 | 10.92345 | 81.0 |
| 4 | 10.003 | 8.83545 | 56.3 |
| 5 | 11.701 | 7.55682 | 37.2 |
| 6 | 12.471 | 7.09202 | 56.2 |
| 7 | 12.941 | 6.83526 | 44.4 |
| 8 | 15.165 | 5.83754 | 52.5 |
| 9 | 16.444 | 5.38629 | 68.9 |
| 10 | 17.432 | 5.08326 | 40.7 |
| 11 | 17.860 | 4.96248 | 17.7 |
| 12 | 19.349 | 4.58380 | 95.4 |
| 13 | 19.998 | 4.43642 | 18.5 |
| 14 | 20.553 | 4.31787 | 86.9 |
| 15 | 21.067 | 4.21361 | 22.4 |
| 16 | 21.709 | 4.09053 | 17.2 |
| 17 | 23.334 | 3.80918 | 6.6 |
| 18 | 24.189 | 3.67638 | 3.1 |
| 19 | 25.772 | 3.45413 | 12.8 |
| 20 | 27.568 | 3.23301 | 13.0 |

### Example 12: Preparation of Crystal Form E of Compound of Formula (I)

Compound of formula (I) (10 mg) was added to DCM (300 uL), dissolved to clarification, then n-heptane (300 uL) was added thereto to precipitate a solid. The mixture was slurried at room temperature and centrifuged. The solid was dried under vacuum to obtain the product. After X-ray powder diffraction detection, the product was defined as crystal form E.

### Example 13: Preparation of Crystal Form F of Compound of Formula (I)

Compound of formula (I) (10 mg) was added to methanol (1.0 mL), slurried at room temperature, and centrifuged. The solid was dried under vacuum to obtain the product. After X-ray powder diffraction detection, the product was defined as crystal form F. The XRPD pattern is shown in FIG. 7, and its characteristic peak positions are shown in Table 6. DSC pattern showed endothermic peaks at 181.22° C. and 245.03° C. TGA pattern showed a weight loss of 8.63% from 25° C. to 210° C.

TABLE 6

| Peak No. | 2θ value [° or degree] | d[Å] | Relative intensity (%) |
|---|---|---|---|
| 1 | 5.062 | 17.44355 | 100.0 |
| 2 | 5.330 | 16.56617 | 24.7 |
| 3 | 7.820 | 11.29642 | 55.0 |
| 4 | 10.077 | 8.77126 | 73.2 |
| 5 | 14.231 | 6.21867 | 31.4 |
| 6 | 15.192 | 5.82720 | 20.3 |
| 7 | 16.672 | 5.31317 | 30.7 |
| 8 | 17.992 | 4.92636 | 9.9 |
| 9 | 18.586 | 4.77014 | 39.8 |
| 10 | 20.435 | 4.34261 | 52.7 |
| 11 | 21.308 | 4.16658 | 11.6 |
| 12 | 21.868 | 4.06116 | 22.7 |
| 13 | 24.193 | 3.67579 | 6.0 |
| 14 | 25.442 | 3.49811 | 15.7 |
| 15 | 26.303 | 3.38548 | 13.6 |
| 16 | 28.629 | 3.11554 | 5.1 |

### Example 14: Preparation of Crystal Form F of Compound of Formula (I)

Compound of formula (I) (10 mg) was added to dichloromethane (0.3 mL), dissolved to clarification, then isopropyl acetate (0.9 mL) was added thereto to precipitate a solid. The mixture was slurried at room temperature and centrifuged. The solid was dried under vacuum to obtain the product. After X-ray powder diffraction detection, the product was defined as crystal form F.

### Example 15: Preparation of Crystal Form F of Compound of Formula (I)

Compound of formula (I) (10 mg) was added to 1,4-dioxane (900 uL) and dissolved to clarification. The mixture was volatilized to obtain the product. After X-ray powder diffraction detection, the product was defined as crystal form F.

### Example 16: Preparation of Crystal Form G of Compound of Formula (I)

Compound of formula (I) (10 mg) was added to 1,2-dichloroethane (1.0 mL), slurried at room temperature, and centrifuged. The solid was dried under vacuum to obtain the product. After X-ray powder diffraction detection, the product was defined as crystal form G. The XRPD pattern is shown in FIG. 8, and its characteristic peak positions are shown in Table 7. DSC pattern showed endothermic peaks at 160.43° C., 182.11° C., and 244.42° C. TGA pattern showed a weight loss of 9.97% from 25° C. to 235° C.

TABLE 7

| Peak No. | 2θ value [° or degree] | d[Å] | Relative intensity (%) |
|---|---|---|---|
| 1 | 5.999 | 14.72062 | 53.4 |
| 2 | 7.396 | 11.94383 | 11.6 |
| 3 | 7.972 | 11.08119 | 18.7 |
| 4 | 8.637 | 10.22906 | 8.9 |
| 5 | 9.951 | 8.88167 | 100.0 |
| 6 | 11.388 | 7.76422 | 19.6 |
| 7 | 12.763 | 6.93064 | 5.3 |
| 8 | 15.291 | 5.78991 | 16.9 |
| 9 | 17.812 | 4.97566 | 66.2 |
| 10 | 18.386 | 4.82162 | 41.3 |
| 11 | 19.105 | 4.64163 | 4.2 |
| 12 | 20.975 | 4.23185 | 31.6 |
| 13 | 22.920 | 3.87702 | 3.9 |
| 14 | 23.408 | 3.79731 | 7.0 |
| 15 | 25.182 | 3.53364 | 5.2 |
| 16 | 25.819 | 3.44790 | 34.4 |
| 17 | 27.400 | 3.25245 | 6.1 |

### Example 17: Preparation of Crystal Form G of Compound of Formula (I)

Compound of formula (I) (10 mg) was added to chloroform (500 uL) and dissolved to clarification. The mixture was volatilized to obtain the product. After X-ray powder diffraction detection, the product was defined as crystal form Gl.

### Example 18: Preparation of Crystal Form H of Compound of Formula (I)

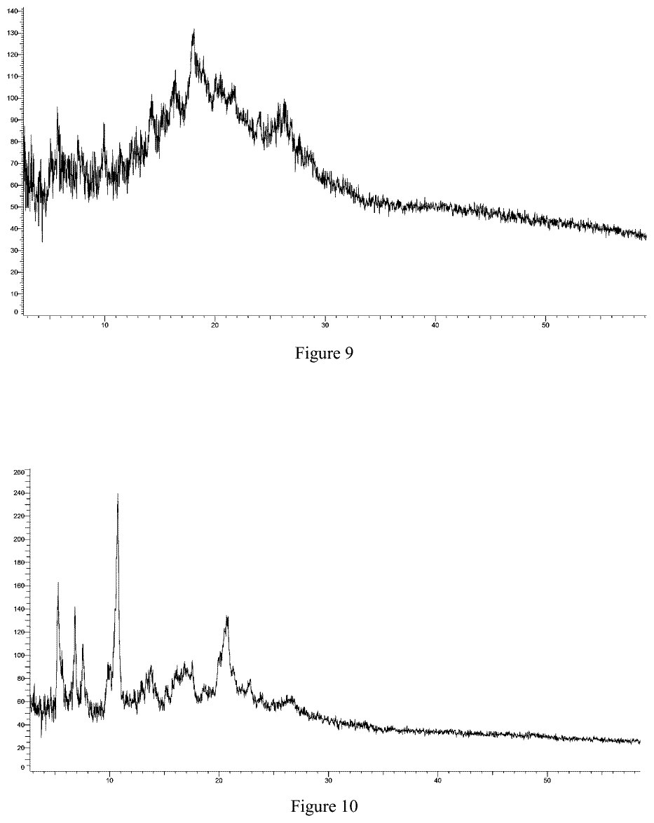

Compound of formula (I) (10 mg) was added to n-hexane (1.0 mL), slurried at room temperature, and centrifuged. The solid was dried under vacuum to obtain the product. After X-ray powder diffraction detection, the product was defined as crystal form H. The XRPD pattern is shown in FIG. 9, and its characteristic peak positions are shown in Table 8. DSC pattern showed endothermic peaks at 184.53° C. and 247.90° C. TGA pattern showed a weight loss of 4.13% from 25° C. to 215° C.

TABLE 8

| Peak No. | 2θ value [° or degree] | d[Å] | Relative intensity (%) |
|---|---|---|---|
| 1 | 5.758 | 15.33508 | 86.9 |
| 2 | 7.533 | 11.72609 | 48.4 |
| 3 | 9.901 | 8.92661 | 88.7 |
| 4 | 14.267 | 6.20293 | 87.7 |
| 5 | 16.420 | 5.39403 | 62.6 |
| 6 | 18.103 | 4.89639 | 100.0 |
| 7 | 18.917 | 4.68744 | 29.4 |
| 8 | 20.489 | 4.33118 | 25.9 |
| 9 | 21.563 | 4.11780 | 2.1 |
| 10 | 24.049 | 3.69743 | 23.1 |
| 11 | 26.356 | 3.37879 | 46.6 |

### Example 19: Preparation of Crystal Form I of Compound of Formula (I)

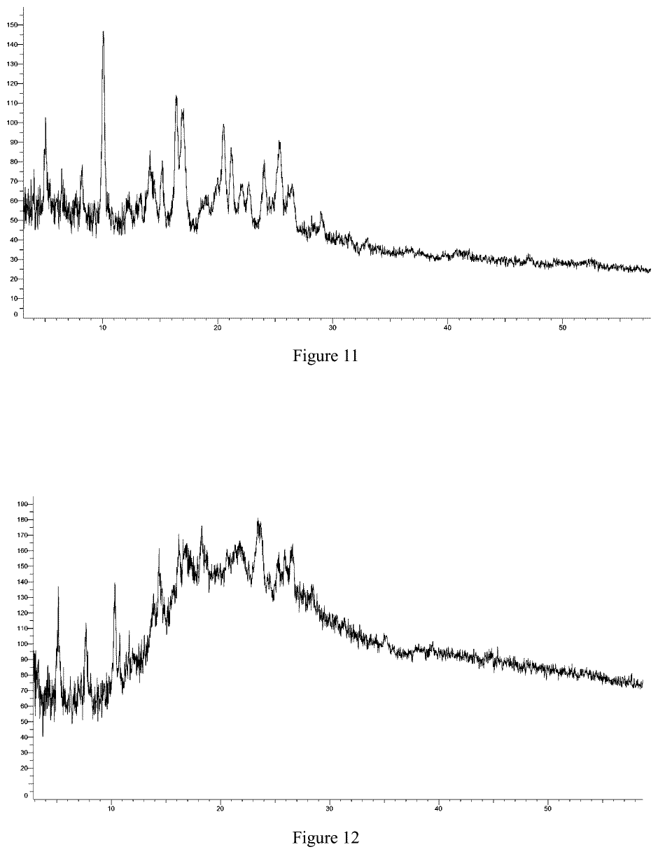

Compound of formula (I) (10 mg) was added to tetrahydrofuran (0.4 mL) and dissolved to clarification. The mixture was slowly volatilizedat room temperature to obtain the product. After X-ray powder diffraction detection, the product was defined as crystal form I. The XRPD pattern is shown in FIG. 10, and its characteristic peak positions are shown in Table 9. DSC pattern showed endothermic peaks at 182.58° C. and 245.04° C. TGA pattern showed a weight loss of 5.44% from 25° C. to 225° C.

TABLE 9

| Peak No. | 2θ value [° or degree] | d[Å] | Relative intensity (%) |
|---|---|---|---|
| 1 | 5.294 | 16.67959 | 57.7 |
| 2 | 6.826 | 12.93958 | 48.2 |
| 3 | 7.564 | 11.67807 | 25.1 |
| 4 | 10.070 | 8.77652 | 19.4 |
| 5 | 10.739 | 8.23157 | 100.0 |
| 6 | 13.699 | 6.45883 | 15.2 |
| 7 | 16.110 | 5.49724 | 9.7 |
| 8 | 16.812 | 5.26944 | 11.9 |
| 9 | 17.601 | 5.03494 | 9.7 |
| 10 | 20.709 | 4.28576 | 31.4 |

### Example 20: Preparation of Crystal Form I of Compound of Formula (I)

Compound of formula (I) (10 mg) was added to tetrahydrofuran (0.3 mL) and dissolved to clarification. The mixture was added with water (0.6 mL) to precipitate a solid, centrifuged, and dried under vacuum to obtain the product. After X-ray powder diffraction detection, the product was defined as crystal form I.

### Example 21: Preparation of Crystal Form J of Compound of Formula (I)

Compound of formula (I) (10 mg) was added to DMSO (0.2 mL) and dissolved to clarification. The mixture was added with water (0.2 mL) to precipitate a solid, centrifuged, and dried under vacuum to obtain the product. After X-ray powder diffraction detection, the product was defined as crystal form J. The XRPD pattern is shown in FIG. 11, and its characteristic peak positions are shown in Table 10. DSC pattern showed endothermic peaks at 154.09° C., 165.41° C., and 247.39° C. TGA pattern showed a weight loss of 10.48% from 30° C. to 220° C.

TABLE 10

| Peak No. | 2θ value [° or degree] | d[Å] | Relative intensity (%) |
|---|---|---|---|
| 1 | 5.041 | 17.51511 | 50.4 |
| 2 | 8.212 | 10.75805 | 25. |
| 3 | 10.068 | 8.77893 | 100.0 |
| 4 | 12.170 | 7.26645 | 6.2 |
| 5 | 14.101 | 6.27581 | 25.3 |
| 6 | 15.167 | 5.83681 | 23.2 |
| 7 | 16.424 | 5.39294 | 58.1 |
| 8 | 16.962 | 5.22293 | 50.0 |
| 9 | 18.862 | 4.70102 | 5.2 |
| 10 | 19.891 | 4.45999 | 4.7 |
| 11 | 20.544 | 4.31982 | 40.3 |
| 12 | 21.190 | 4.18956 | 27.3 |
| 13 | 22.036 | 4.03053 | 9.6 |
| 14 | 22.679 | 3.91762 | 12.8 |
| 15 | 24.077 | 3.69323 | 27.5 |
| 16 | 25.433 | 3.49933 | 36.0 |
| 17 | 26.111 | 3.41003 | 14.4 |
| 18 | 26.454 | 3.36657 | 14.6 |
| 19 | 28.942 | 3.08259 | 6.2 |

### Example 22: Preparation of Crystal Form J of Compound of Formula (I)

Compound of formula (I) (10 mg) was added to DMSO (0.2 mL) and dissolved to clarification. The mixture was added with methyl tert-butyl ether (0.4 mL) to precipitate a solid, centrifuged, and dried under vacuum to obtain the product. After X-ray powder diffraction detection, the product was defined as crystal form J.

### Example 23: Preparation of Crystal Form K of Compound of Formula (I)

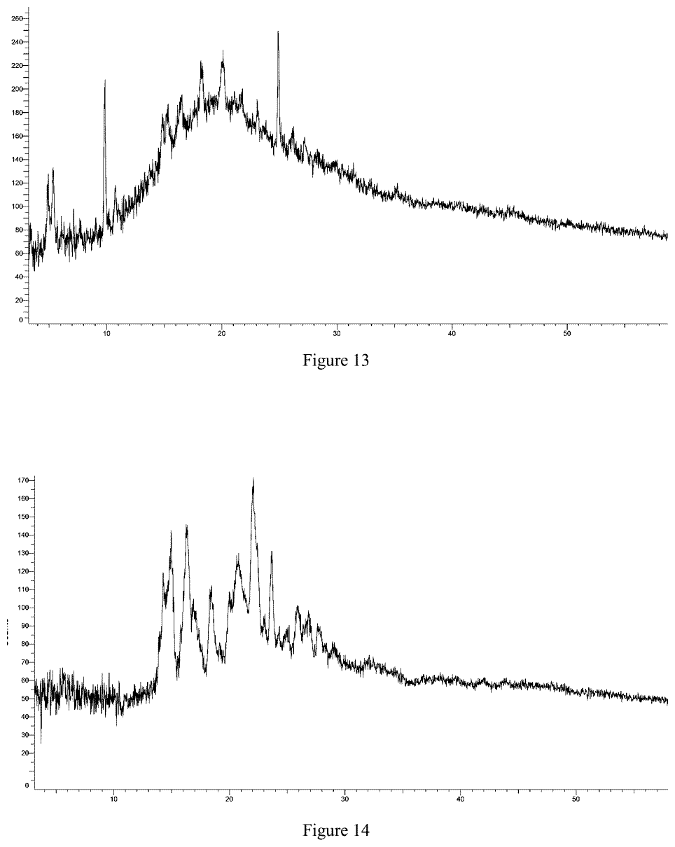

Compound of formula (I) (10 mg) was added to N-methylpyrrolidone (50 uL) and dissolved to clarification. The mixture was slowly volatilizedat room temperature to obtain the product. After X-ray powder diffraction detection, the product was defined as crystal form K. The XRPD pattern is shown in FIG. 12, and its characteristic peak positions are shown in Table 11

TABLE 11

| Peak No. | 2θ value [° or degree] | d[Å] | Relative intensity (%) |
|---|---|---|---|
| 1 | 5.156 | 17.12608 | 100.0 |
| 2 | 7.699 | 11.47378 | 66.7 |
| 3 | 10.339 | 8.54876 | 82.2 |
| 4 | 10.735 | 8.23452 | 25.6 |
| 5 | 13.886 | 6.37246 | 33.2 |
| 6 | 14.334 | 6.17431 | 46.9 |
| 7 | 16.203 | 5.46577 | 43.1 |
| 8 | 18.327 | 4.83696 | 33.3 |
| 9 | 23.418 | 3.79575 | 35.9 |
| 10 | 23.725 | 3.74729 | 27.9 |
| 11 | 25.348 | 3.51082 | 18.0 |
| 12 | 25.919 | 3.43482 | 25.7 |
| 13 | 26.446 | 3.36761 | 22.8 |

### Example 24: Preparation of Crystal Form L of Compound of Formula (I)

Compound of formula (I) (10 mg) was added to N,N-dimethylacetamide (200 uL). After the mixture turned clear, it was allowed to slowly evaporate under room temperature to obtain the product. After X-ray powder diffraction detection, the product was defined as crystal form L. The XRPD pattern is shown in FIG. 13, and its characteristic peak positions are shown in Table 12.

TABLE 12

| | 2θ value | | Relative |
|---|---|---|---|
| Peak No. | [° or degree] | d[Å] | intensity (%) |
| 1 | 4.932 | 17.90379 | 53.0 |
| 2 | 5.360 | 16.47277 | 59.3 |
| 3 | 9.831 | 8.98960 | 100.0 |
| 4 | 10.753 | 8.22126 | 27.2 |
| 5 | 14.844 | 5.96326 | 31.7 |
| 6 | 16.369 | 5.41103 | 18.0 |
| 7 | 18.244 | 4.85878 | 28.9 |
| 8 | 20.104 | 4.41318 | 39.6 |
| 9 | 23.129 | 3.84244 | 6.8 |
| 10 | 24.914 | 3.57098 | 75.6 |

### Example 25: Preparation of Crystal Form M of Compound of Formula (I)

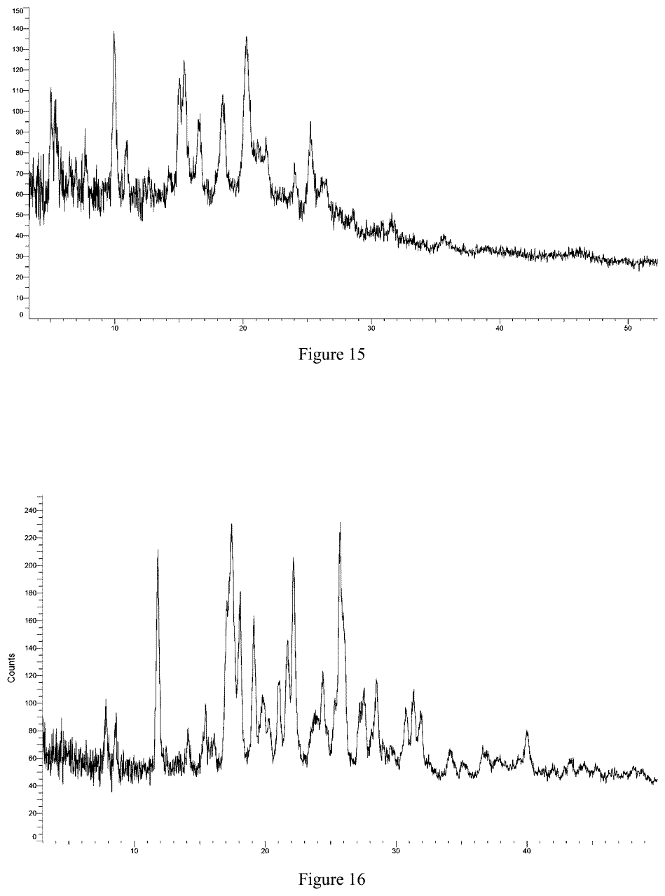

The crystal form A of the compound of formula (I) (10 mg) was heated to 225° C. to obtain the product. After X-ray powder diffraction detection, the product was defined as crystal form M. The XRPD pattern is shown in FIG. 14, and its characteristic peak positions are shown in Table 13.

TABLE 13

| | 2θ value | | Relative |
|---|---|---|---|
| Peak No. | [° or degree] | d[Å] | intensity (%) |
| 1 | 14.202 | 6.23131 | 48.9 |
| 2 | 14.959 | 5.91770 | 95.7 |
| 3 | 16.322 | 5.42636 | 86.5 |
| 4 | 18.410 | 4.81529 | 44.0 |
| 5 | 20.016 | 4.43252 | 27.8 |
| 6 | 20.748 | 4.27773 | 48.6 |
| 7 | 22.067 | 4.02498 | 100.0 |
| 8 | 23.670 | 3.75578 | 54.6 |
| 9 | 24.322 | 3.65655 | 1.7 |
| 10 | 24.839 | 3.58163 | 1.7 |
| 11 | 25.873 | 3.44085 | 17.3 |
| 12 | 26.863 | 3.31618 | 20.0 |
| 13 | 27.811 | 3.20532 | 12.5 |

### Example 26: Preparation of Crystal Form N of Compound of Formula (I)

Compound of formula (I) (80 mg) was added to a mixed solvent of ethyl acetate and tetrahydrofuran (4.0 mL, V/V=1:1). The mixture was heated and dissolved to clarification, recrystallized, and filtered. The resulting solid was dried to obtain the product. After X-ray powder diffraction detection, the product was defined as crystal form N. The XRPD pattern is shown in FIG. 15, and its characteristic peak positions are shown in Table 14. DSC pattern showed endothermic peaks at 185.49° C. and 188.82° C. TGA pattern showed a weight loss of 4.86% from 30° C. to 225° C.

TABLE 14

| | 2θ value | | Relative |
|---|---|---|---|
| Peak No. | [° or degree] | d[Å] | intensity (%) |
| 1 | 5.028 | 17.56063 | 74.1 |
| 2 | 7.671 | 11.51522 | 35.7 |
| 3 | 9.942 | 8.88987 | 100.0 |
| 4 | 10.900 | 8.11071 | 37.1 |
| 5 | 12.677 | 6.97719 | 17.6 |
| 6 | 15.046 | 5.88374 | 68.9 |
| 7 | 15.428 | 5.73862 | 74.8 |
| 8 | 16.560 | 5.34887 | 35.0 |
| 9 | 18.410 | 4.81526 | 56.6 |
| 10 | 20.274 | 4.37654 | 90.8 |
| 11 | 21.148 | 4.19761 | 24.0 |
| 12 | 21.765 | 4.08016 | 25.3 |
| 13 | 24.036 | 3.69941 | 18.6 |
| 14 | 25.252 | 3.52401 | 51.6 |
| 15 | 26.385 | 3.37516 | 17.3 |

### Example 27: Preparation of Crystal Form O of Compound of Formula (I)

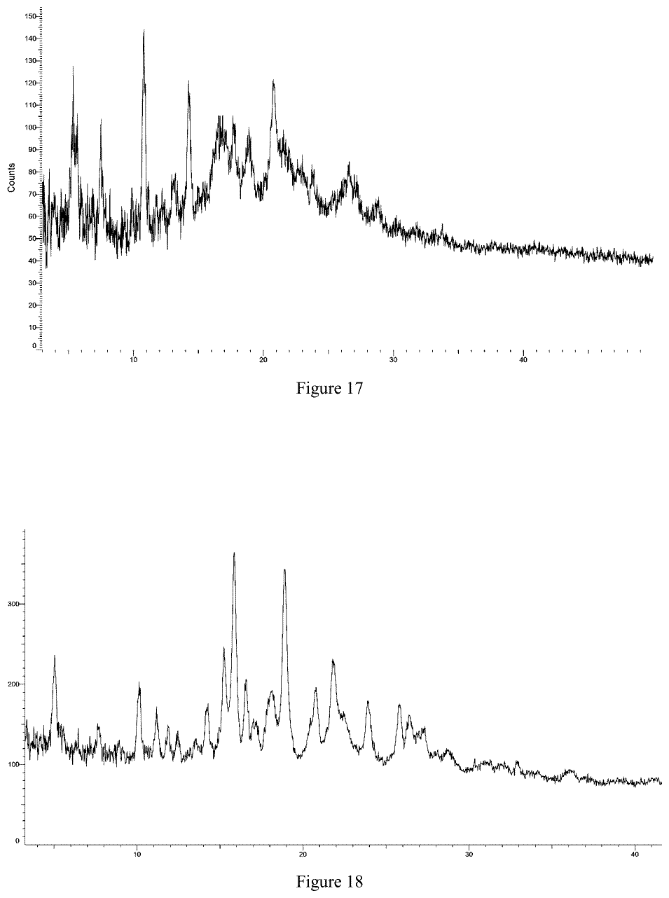

Compound of formula (I) (60 mg) was added to acetonitrile (5 mL). Then benzenesulfonic acid (16 mg) was added thereto at room temperature, and the reaction mixture was reacted at 80° C. for 8 hours. A solid was precipitated out of the reaction mixture, filtered, and then dried at 45° C. under vacuum to obtain the product. After X-ray powder diffraction detection, the product was defined as crystal form O. The XRPD pattern is shown in FIG. 16, and its characteristic peak positions are shown in Table 15.

TABLE 15

| | 2θ value | | Relative |
|---|---|---|---|
| Peak No. | [° or degree] | d[Å] | intensity (%) |
| 1 | 7.843 | 11.26283 | 31.3 |
| 2 | 8.558 | 10.32402 | 23.0 |
| 3 | 11.796 | 7.49634 | 92.7 |
| 4 | 14.106 | 6.27359 | 15.1 |
| 5 | 15.455 | 5.72872 | 24.5 |
| 6 | 16.088 | 5.50482 | 11.8 |
| 7 | 17.082 | 5.18651 | 67.4 |
| 8 | 17.423 | 5.08573 | 100.0 |
| 9 | 18.081 | 4.90224 | 67.1 |
| 10 | 19.136 | 4.63424 | 58.7 |
| 11 | 19.858 | 4.46735 | 23.4 |
| 12 | 20.281 | 4.37519 | 13.6 |
| 13 | 21.055 | 4.21593 | 29.3 |
| 14 | 21.707 | 4.09087 | 46.9 |
| 15 | 22.165 | 4.00735 | 81.0 |
| 16 | 23.743 | 3.74453 | 17.7 |
| 17 | 24.412 | 3.64329 | 33.9 |
| 18 | 25.719 | 3.46112 | 98.6 |
| 19 | 25.934 | 3.43290 | 57.0 |
| 20 | 27.212 | 3.27445 | 21.5 |
| 21 | 27.538 | 3.23639 | 24.1 |
| 22 | 28.521 | 3.12710 | 30.6 |
| 23 | 30.766 | 2.90380 | 21.3 |
| 24 | 31.329 | 2.85290 | 27.4 |
| 25 | 31.868 | 2.80590 | 21.1 |
| 26 | 34.061 | 2.63007 | 8.1 |
| 27 | 36.625 | 2.45162 | 8.4 |
| 28 | 39.995 | 2.25249 | 13.8 |

### Example 28: Preparation of Crystal Form P of Compound of Formula (I)

The crystal form C of the compound of formula (I) (40 mg) was added to dioxane (2.0 mL), slurried at room temperature, and filtered. The solid was dried under vacuum to obtain the product. After X-ray powder diffraction detection, the product was defined as crystal form P. The XRPD pattern is shown in FIG. 17. DSC pattern showed endothermic peaks at 127.49° C., 180.10° C., and 249.23° C. TGA pattern showed a weight loss of 8.79% from 40° C. to 230° C.

TABLE 16

| | 2θ value | | Relative |
|---|---|---|---|
| Peak No. | [° or degree] | d[Å] | intensity (%) |
| 1 | 5.359 | 16.47692 | 86.4 |
| 2 | 5.629 | 15.68836 | 53.7 |
| 3 | 7.491 | 11.79123 | 57.4 |
| 4 | 9.905 | 8.92296 | 22.1 |
| 5 | 10.786 | 8.19548 | 100.0 |
| 6 | 13.192 | 6.70583 | 20.4 |
| 7 | 14.249 | 6.21072 | 63.1 |
| 8 | 16.527 | 5.35944 | 35.1 |
| 9 | 17.729 | 4.99885 | 25.8 |
| 10 | 18.862 | 4.70104 | 16.6 |
| 11 | 20.798 | 4.26747 | 45.3 |
| 12 | 23.799 | 3.73579 | 9.8 |
| 13 | 26.555 | 3.35399 | 16.6 |

### Example 27: Preparation of Crystal Form Q of Compound of Formula (I)

Compound of formula (I) (150 mg) was added to ethyl acetate (3 mL), slurried at room temperature, and filtered. The solid was dried at 130° C. under vacuum to obtain the product. After X-ray powder diffraction detection, the product was defined as crystal form Q. The XRPD pattern is shown in FIG. 18, and its characteristic peak positions are shown in Table 17. DSC pattern showed endothermic peaks at 188.58° C. and 249.61° C. TGA pattern showed a weight loss of 0.54% from 25° C. to 225° C.

TABLE 17

| | 20 value | | Relative |
|---|---|---|---|
| Peak No. | [° or degree] | d[Å] | intensity (%) |
| 1 | 5.038 | 17.52734 | 49.1 |
| 2 | 7.682 | 11.49962 | 14.1 |
| 3 | 10.152 | 8.70606 | 30.2 |
| 4 | 11.175 | 7.91146 | 22.0 |
| 5 | 11.902 | 7.42999 | 13.2 |
| 6 | 12.461 | 7.09750 | 12.0 |
| 7 | 13.544 | 6.53264 | 7.0 |
| 8 | 14.218 | 6.22410 | 20.4 |
| 9 | 15.246 | 5.80700 | 51.2 |
| 10 | 15.850 | 5.58692 | 100.0 |
| 11 | 16.574 | 5.34448 | 32.9 |
| 12 | 17.012 | 5.20787 | 11.5 |
| 13 | 18.124 | 4.89081 | 27.5 |
| 14 | 18.892 | 4.69354 | 92.2 |
| 15 | 20.760 | 4.27525 | 28.5 |
| 16 | 21.835 | 4.06714 | 42.5 |
| 17 | 22.440 | 3.95884 | 17.6 |
| 18 | 23.905 | 3.71949 | 24.8 |
| 19 | 25.784 | 3.45248 | 25.2 |
| 20 | 26.418 | 3.37105 | 19.2 |
| 21 | 27.295 | 3.26466 | 8.1 |

### Example 29: Preparation of Crystal Form U of Compound of Formula (I)

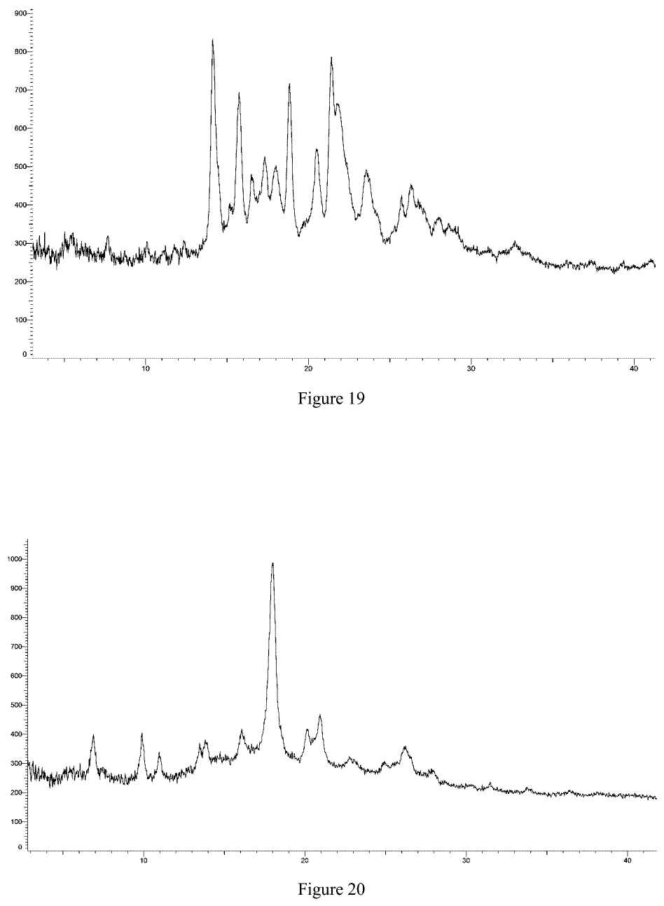

Compound of formula (I) (50 mg) was added to DMSO (0.6 mL). The mixture was heated at 50° C. and dissolved to clarification, then added with water (1.2 mL) for crystallization, and then centrifuged. The solid was washed with an additional 2 mL of water, centrifuged, and dried under vacuum to obtain the product. After X-ray powder diffraction detection, the product was defined as crystal form U. The XRPD pattern is shown in FIG. 19, and its characteristic peak positions are shown in Table 18. DSC pattern showed endothermic peaks at 183.28° C. and 248.22° C. TGA pattern showed a weight loss of 0.62% from 40° C. to 215° C.

TABLE 18

| | 2θ value | | Relative |
|---|---|---|---|
| Peak No. | [° or degree] | d[Å] | intensity (%) |
| 1 | 7.657 | 11.53595 | 10.5 |
| 2 | 14.155 | 6.25201 | 100.0 |
| 3 | 15.745 | 5.62385 | 70.6 |
| 4 | 16.564 | 5.34760 | 25.9 |
| 5 | 17.314 | 5.11762 | 35.7 |
| 6 | 17.997 | 4.92481 | 31.1 |
| 7 | 18.838 | 4.70701 | 73.5 |
| 8 | 20.512 | 4.32631 | 38.7 |
| 9 | 21.415 | 4.14595 | 85.4 |
| 10 | 21.768 | 4.07959 | 61.7 |
| 11 | 23.557 | 3.77361 | 31.0 |
| 12 | 25.711 | 3.46206 | 19.3 |
| 13 | 26.313 | 3.38426 | 23.5 |
| 14 | 28.029 | 3.18080 | 9.9 |

### Example 30: Preparation of Crystal Form U of Compound of Formula (I)

The crystal form Q of the compound of formula (I) (10 mg) was added to water (1.0 mL), and slurried at room temperature for 1 day to obtain the product. After X-ray powder diffraction detection, the product was defined as crystal form U.

### Example 31: Preparation of Crystal Form X of Compound of Formula (I)

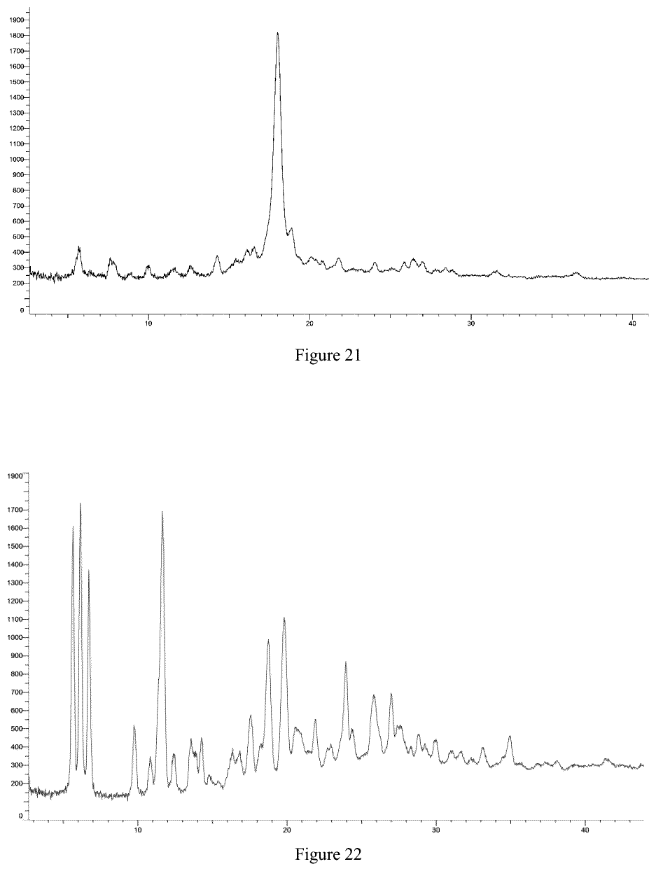

The crystal form D of the compound of formula (I) (10 mg) was added to n-heptane (1.0 mL), and the mixture was slurried at room temperature and centrifuged. The solid was dried under vacuum to obtain the product. After X-ray powder diffraction detection, the product was defined as crystal form X. The XRPD pattern is shown in FIG. 20, and its characteristic peak positions are shown in Table 19. DSC pattern showed endothermic peaks at 158.81° C., 184.14° C., and 249.04° C.

TABLE 19

| | 20 value | | Relative |
|---|---|---|---|
| Peak No. | [° or degree] | d[Å] | intensity (%) |
| 1 | 6.864 | 12.86741 | 21.8 |
| 2 | 9.873 | 8.95188 | 22.0 |
| 3 | 10.963 | 8.06358 | 12.8 |
| 4 | 13.475 | 6.56575 | 12.5 |
| 5 | 13.801 | 6.41151 | 13.3 |
| 6 | 16.089 | 5.50446 | 14.7 |
| 7 | 18.006 | 4.92253 | 100.0 |
| 8 | 20.158 | 4.40145 | 15.4 |
| 9 | 20.929 | 4.24113 | 23.1 |
| 10 | 26.203 | 3.39818 | 12.2 |

### Example 32: Preparation of Crystal Form of Compound of Formula (I)

The crystal form Q of the compound of formula (I) (10 mg) was added to n-heptane (1.0 mL), slurried at room temperature, and centrifuged. The solid was dried under vacuum to obtain the product. After X-ray powder diffraction detection, the product was defined as crystal form Y The XRPD pattern is shown in FIG. 21, and its characteristic peak positions are shown in Table 20. DSC pattern showed endothermic peaks at 184.97° C. and 248.72° C.

TABLE 20

| | 2θ value | | Relative |
|---|---|---|---|
| Peak No. | [° or degree] | d[Å] | intensity (%) |
| 1 | 5.676 | 15.55652 | 13.3 |
| 2 | 7.666 | 11.52267 | 7.1 |
| 3 | 7.875 | 11.21711 | 6.2 |
| 4 | 9.985 | 8.85146 | 3.5 |
| 5 | 12.634 | 7.00073 | 3.9 |
| 6 | 14.260 | 6.20594 | 7.5 |
| 7 | 16.114 | 5.49605 | 6.8 |
| 8 | 16.562 | 5.34817 | 7.8 |
| 9 | 18.020 | 4.91859 | 100.0 |
| 10 | 18.874 | 4.69807 | 14.9 |
| 11 | 21.802 | 4.07319 | 4.5 |
| 12 | 24.051 | 3.69713 | 3.1 |
| 13 | 25.846 | 3.44430 | 3.2 |
| 14 | 26.425 | 3.37019 | 4.9 |
| 15 | 26.974 | 3.30277 | 3.6 |

### Example 33: Preparation of Crystal Form V of Compound of Formula (I)

Compound of formula (I) (200 mg) was added to N,N-dimethylformamide (0.4 mL). Then acetonitrile (2 mL) was added to the system. The mixture was stirred at 20° C. to 30° C., filtered under reduced pressure. The filter cake was dried to obtain the product. After X-ray powder diffraction detection, the product was defined as crystal form V. The XRPD pattern is shown in FIG. 22, and its characteristic peak positions are shown in Table 21. DSC pattern showed an endothermic peak at 147.78° C. TGA pattern showed a weight loss of 10.73% from 30° C. to 210° C.

TABLE 21

| | 2θ value | | Relative |
|---|---|---|---|
| Peak No. | [° or degree] | d[Å] | intensity (%) |
| 1 | 5.649 | 15.63089 | 90.2 |
| 2 | 6.154 | 14.34928 | 100.0 |
| 3 | 6.720 | 13.14362 | 77.8 |
| 4 | 9.778 | 9.03863 | 23.2 |
| 5 | 10.830 | 8.16267 | 12.3 |
| 6 | 11.409 | 7.74959 | 38.9 |
| 7 | 11.651 | 7.58921 | 96.2 |
| 8 | 12.390 | 7.13839 | 12.9 |
| 9 | 13.576 | 6.51708 | 17.6 |
| 10 | 13.878 | 6.37593 | 12.9 |
| 11 | 14.272 | 6.20085 | 17.1 |
| 12 | 14.769 | 5.99321 | 4.1 |
| 13 | 16.361 | 5.41358 | 11.8 |
| 14 | 16.815 | 5.26837 | 8.6 |
| 15 | 17.570 | 5.04354 | 20.6 |
| 16 | 18.274 | 4.85076 | 9.8 |
| 17 | 18.757 | 4.72706 | 45.0 |
| 18 | 19.813 | 4.47745 | 51.3 |
| 19 | 20.609 | 4.30614 | 13.0 |
| 20 | 20.727 | 4.28207 | 12.2 |
| 21 | 21.905 | 4.05427 | 15.5 |
| 22 | 22.721 | 3.91056 | 5.2 |
| 23 | 22.947 | 3.87250 | 6.0 |
| 24 | 23.948 | 3.71286 | 33.0 |
| 25 | 24.370 | 3.64956 | 9.2 |
| 26 | 25.825 | 3.44713 | 20.2 |
| 27 | 26.995 | 3.30028 | 20.3 |
| 28 | 27.595 | 3.22984 | 9.5 |
| 29 | 28.859 | 3.09128 | 7.2 |
| 30 | 29.953 | 2.98076 | 5.9 |
| 31 | 31.628 | 2.82660 | 3.1 |
| 32 | 33.136 | 2.70136 | 5.3 |
| 33 | 34.931 | 2.56651 | 10.0 |

### Example 34: Preparation of Crystal Form V of Compound of Formula (I)

Compound of formula (I) (200 mg) was added to N,N-dimethylformamide (1 mL). The mixture was heated to 60° C. until the compound was dissolved, then cooled naturally to 20° C.-30° C., stirred at 20° C.-30° C., and filtered under reduced pressure. The filter cake was rinsed with N,N-dimethylformamide (0.2 mL), and dried at 45° C. under reduced pressure for 26 hours to obtain the product. After X-ray powder diffraction detection, the product was defined as crystal form V.

### Example 35: Preparation of Crystal Form R of Compound of Formula (I)

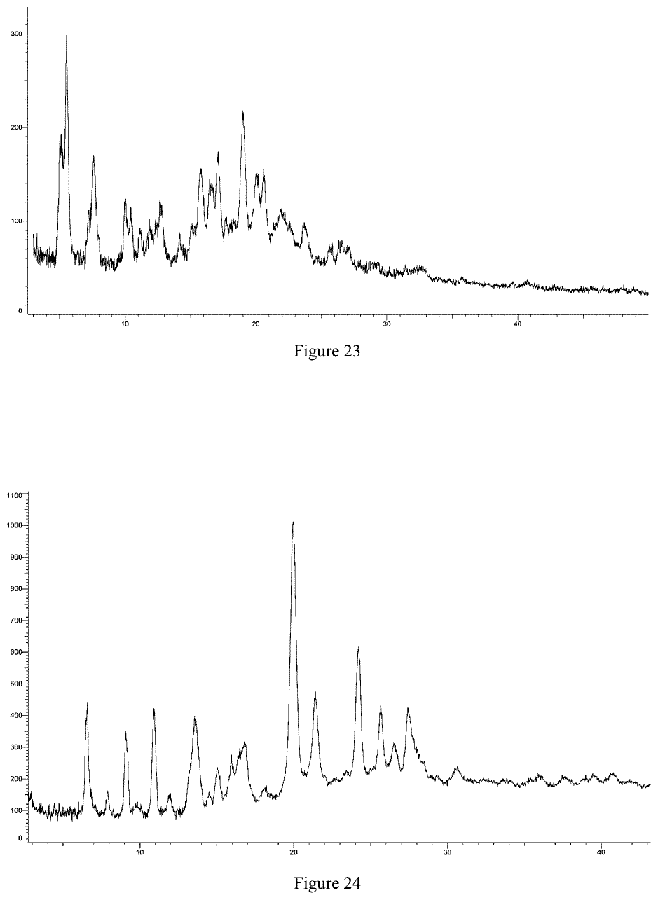

Compound of formula (I) (8 g) was added to tetrahydrofuran (150 mL). The mixture was stirred until the compound was dissolved, and concentrated under reduced pressure until no distillate appeared, thus obtaining the product. After X-ray powder diffraction detection, the product was defined as crystal form R. The XRPD pattern is shown in FIG. 23, and its characteristic peak positions are shown in Table 22.

TABLE 22

| | 2θ value | | Relative |
|---|---|---|---|
| Peak No. | [° or degree] | d[Å] | intensity (%) |
| 1 | 5.132 | 17.20688 | 57.2 |
| 2 | 5.534 | 15.95544 | 100.0 |
| 3 | 7.232 | 12.21394 | 23.7 |
| 4 | 7.611 | 11.60654 | 48.8 |
| 5 | 7.831 | 11.28001 | 29.7 |
| 6 | 10.033 | 8.80950 | 26.8 |
| 7 | 10.437 | 8.46890 | 18.1 |
| 8 | 11.148 | 7.93072 | 12.0 |
| 9 | 11.857 | 7.45771 | 16.1 |
| 10 | 12.737 | 6.94459 | 20.8 |
| 11 | 14.179 | 6.24131 | 10.5 |
| 12 | 15.782 | 5.61066 | 31.9 |
| 13 | 16.575 | 5.34421 | 24.6 |
| 14 | 17.101 | 5.18101 | 36.3 |
| 15 | 19.017 | 4.66306 | 51.1 |
| 16 | 20.073 | 4.42007 | 26.4 |
| 17 | 20.567 | 4.31493 | 27.9 |
| 18 | 21.871 | 4.06049 | 12.5 |
| 19 | 23.692 | 3.75232 | 12.7 |

### Example 36: Preparation of Crystal Form S of Compound of Formula (I)

Compound of formula (I) was dissolved in N,N-dimethylformamide by heating, then precipitated by cooling. The compound was slurried with acetonitrile, and then filtered. The filter cake was slurried with water once, and filtered. The sample was dried at 60° C., added with acetonitrile (1 L), stirred at 20° C.-30° C., filtered, and dried to obtain the product. After X-ray powder diffraction detection, the product was defined as crystal form S. The XRPD pattern is shown in FIG. 24, and its characteristic peak positions are shown in Table 23.

TABLE 23

| | 2θ value | | Relative |
|---|---|---|---|
| Peak No. | [° or degree] | d[Å] | intensity (%) |
| 1 | 6.576 | 13.42968 | 41.0 |
| 2 | 7.890 | 11.19569 | 8.5 |
| 3 | 9.082 | 9.72923 | 31.3 |
| 4 | 10.921 | 8.09450 | 39.0 |
| 5 | 11.921 | 7.41792 | 7.3 |
| 6 | 13.592 | 6.50936 | 31.5 |
| 7 | 14.500 | 6.10405 | 4.3 |
| 8 | 15.043 | 5.88470 | 12.5 |
| 9 | 15.951 | 5.55172 | 16.3 |
| 10 | 16.495 | 5.36979 | 18.6 |
| 11 | 16.805 | 5.27157 | 20.9 |
| 12 | 19.965 | 4.44366 | 100.0 |
| 13 | 21.403 | 4.14833 | 34.5 |
| 14 | 24.207 | 3.67365 | 46.0 |
| 15 | 25.662 | 3.46867 | 23.7 |
| 16 | 26.537 | 3.35624 | 9.2 |
| 17 | 27.457 | 3.24575 | 21.5 |
| 18 | 30.646 | 2.91489 | 4.8 |

### Example 37: Preparation of Crystal Form T of Compound of Formula (I)

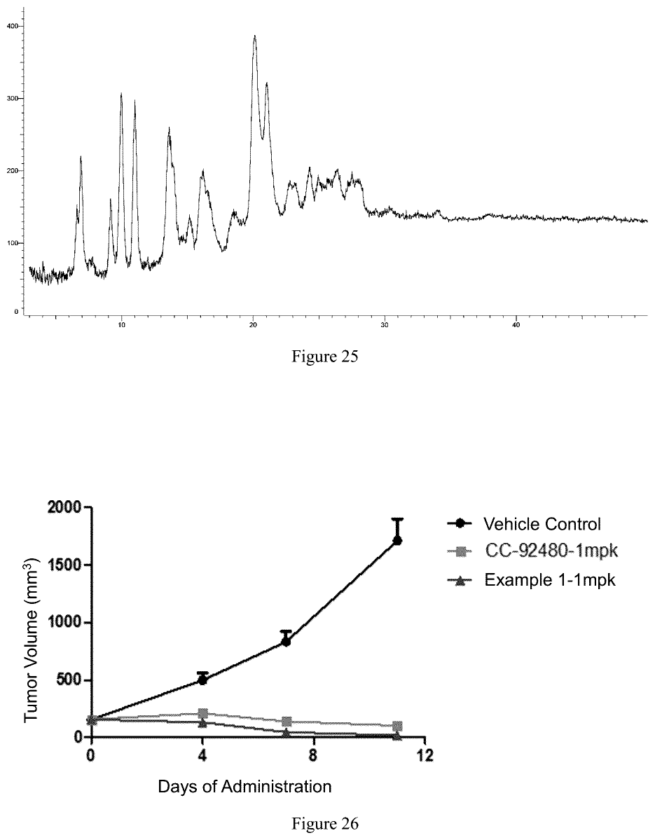

Compound of formula (I) (70.9 g) was added to N,N-dimethylformamide (200 mL). The mixture was heated to 70° C.-80° C. until the compound was dissolved, then cooled to 20° C.-30° C. to precipitate the compound, stirred, and filtered, and the filter cake was added to water (1200 mL) and stirred. After filtering, the filter cake was again added to water (1200 mL) and stirred, followed by filtration. The filter cake was dried at 40° C.-45° C. The dried material (45.0 g) was added to acetonitrile (450 mL), and subjected to magnetic stirring at 20° C.-30° C. for 29 hours. The mixture was filtered under reduced pressure, then dried under reduced pressure for 17 hours to obtain the product. After X-ray powder diffraction detection, the product was defined as crystal form T. The XRPD pattern is shown in FIG. 25, and its characteristic peak positions are shown in Table 24. DSC pattern showed endothermic peaks at 164.78° C. and 247.66° C.

TABLE 24

| | 2θ value | | Relative |
|---|---|---|---|
| Peak No. | [° or degree] | d[Å] | intensity (%) |
| 1 | 6.610 | 13.36086 | 37.5 |
| 2 | 6.915 | 12.77296 | 62.8 |
| 3 | 9.177 | 9.62879 | 37.7 |
| 4 | 9.984 | 8.85256 | 94.9 |
| 5 | 11.012 | 8.02791 | 87.7 |
| 6 | 13.595 | 6.50797 | 65.3 |
| 7 | 13.926 | 6.35399 | 45.7 |
| 8 | 15.174 | 5.83405 | 13.8 |
| 9 | 16.156 | 5.48189 | 38.6 |
| 10 | 18.509 | 4.78977 | 12.4 |
| 11 | 20.138 | 4.40594 | 100.0 |
| 12 | 21.037 | 4.21952 | 70.8 |
| 13 | 22.954 | 3.87131 | 7.4 |
| 14 | 24.261 | 3.66563 | 16.7 |
| 15 | 26.391 | 3.37441 | 13.3 |
| 16 | 27.514 | 3.23919 | 10.0 |

### Example 38: Hygroscopicity Study of Crystal Forms D, E, Q, and U of

Compound of Formula (I) (Oct. 15, 2021, Nanjing) 03881 Using the Surface Measurement Systems advantage 2, at a temperature of 25° C., and starting at a humidity of 50%, the humidity range was examined from 000 to 95%, with increments of 10%. The criterion for evaluation was that the mass change dM/dT at each gradient was less than 0.002%, TMAX was 360 min, and the process was cycled twice.

TABLE 25

| Test | | | |
|---|---|---|---|
| sample | 0.0% RH-95.0% RH | 20% RH-80% RH | Crystal form |
| Crystal | 2.24% | 1.26% | Untransformed |
| form D | | (slightly hygroscopic) | |
| Crystal | 2.24% | 0.84% | Untransformed |
| form E | | (slightly hygroscopic) | |
| Crystal | 0.67% | 0.24% | Untransformed |
| form Q | | (slightly hygroscopic) | |
| Crystal | 2.06% | 1.03% | Untransformed |
| form U | | (slightly hygroscopic) | |

### Example 39: Stability of Factors Influencing Crystal Forms D, E, Q, and U of Compound of Formula (I)

Each crystal form was spread out in an open exposure setup, and the stability of the samples under conditions of light (4500 Lux), high temperature (40° C., 60° C.), and high humidity (RH 75 RH 92.5%) was examined separately. The sampling inspection period was 30 days.

TABLE 26

| | | | Free crystal form D | | |
|---|---|---|---|---|---|
| | | Time | Color and | | Crystal |
| Condition | | (days) | character | Purity % | form |
| Onset | | 0 | White solid | 97.9 | D |
| 40° | C. | 9 | White solid | 97.3 | D |
| | | 15 | White solid | 96.7 | D |
| | | 30 | White solid | 95.8 | D |
| 60° | C. | 9 | White solid | 96.6 | D |
| | | 15 | White solid | 95.8 | D |
| | | 30 | White solid | 93.9 | D |
| 75% | RH | 9 | White solid | 97.8 | D |
| | | 15 | White solid | 97.7 | D |
| | | 30 | White solid | 97.9 | D |
| 92.5% | RH | 9 | White solid | 97.8 | D |
| | | 15 | White solid | 97.9 | D |
| | | 30 | White solid | 97.8 | D |
| 4500 | Lux | 9 | White solid | 97.3 | D |
| | | 15 | White solid | 96.8 | D |
| | | 30 | White solid | 95.4 | D |
| | | | Free crystal form E | | |
| | | Time | Color and | | Crystal |
| Condition | | (days) | character | Purity % | form |
| Onset | | 0 | White solid | 97.9 | E |
| 40° | C. | 9 | White solid | 97.4 | E |
| | | 15 | White solid | 97.0 | E |
| | | 30 | White solid | 96.4 | E |
| 60° | C. | 9 | White solid | 96.7 | E |
| | | 15 | White solid | 95.9 | E |
| | | 30 | White solid | 94.6 | E |
| 75% | RH | 9 | White solid | 97.9 | E |
| | | 15 | White solid | 97.9 | E |
| | | 30 | White solid | 97.8 | E |
| 92.5% | RH | 9 | White solid | 97.9 | E |
| | | 15 | White solid | 97.8 | E |
| | | 30 | White solid | 97.8 | E |
| 4500 | Lux | 9 | White solid | 97.7 | E |
| | | 15 | White solid | 97.4 | E |
| | | 30 | White solid | 94.9 | E |
| | | | Free crystal form Q | | |
| | | Time | Color and | | Crystal |
| Condition | | (days) | character | Purity % | form |
| Onset | | 0 | White solid | 99.6 | Q |
| 40° | C. | 7 | White solid | 99.5 | Q |
| | | 14 | White solid | 99.4 | Q |
| | | 30 | White solid | 99.1 | Q |
| 60° | C. | 7 | White solid | 99.3 | Q |
| | | 14 | White solid | 98.9 | Q |
| | | 30 | White solid | 98.5 | Q |
| 75% | RH | 7 | White solid | 99.6 | Q |
| | | 14 | White solid | 99.6 | Q |
| | | 30 | White solid | 99.6 | Q |
| 92.5% | RH | 14 | White solid | 99.6 | Q |
| | | 30 | White solid | 99.5 | Q |
| 4500 | Lux | 7 | White solid | 99.5 | Q |
| | | 14 | White solid | 98.8 | Q |
| | | 30 | White solid | 93.9 | Q |
| | | | Free crystal form U | | |
| | | Time | Color and | | Crystal |
| Condition | | (days) | character | Purity % | form |
| Onset | | 0 | White solid | 99.7 | U |
| 40° | C. | 7 | White solid | 99.5 | U |
| | | 14 | White solid | 99.3 | U |
| | | 30 | White solid | 98.8 | U |
| 60° | C. | 7 | White solid | 98.9 | U |
| | | 14 | White solid | 98.5 | U |
| | | 30 | White solid | 97.5 | U |
| 75% | RH | 7 | White solid | 99.6 | U |
| | | 14 | White solid | 99.7 | U |
| | | 30 | White solid | 99.6 | U |
| 92.5% | RH | 7 | White solid | 99.7 | U |
| | | 14 | White solid | 99.7 | U |
| | | 30 | White solid | 99.6 | U |
| 4500 | Lux | 7 | White solid | 99.3 | U |
| | | 14 | White solid | 99.3 | U |
| | | 30 | White solid | 98.8 | U |

Conclusion: The influencing factor experiments demonstrate that the crystal forms D, E, Q, and U of the compound of formula (I) exhibit good physicochemical stability under high humidity conditions of 75% and 92.5%. There is a slight decrease in purity under the conditions of 60° C. and light, and the crystal forms remain unchanged.

### Example 40: Long-Term/Accelerated Stability of Crystal Forms D, E, Q, and U of Compound of Formula (I)

The crystal forms D, E, Q, and U of the compound of formula (I) were respectively placed under conditions of 25° C. with 60% RH and 40° C. with 75% RH to evaluate their stability.

TABLE 27

| | | | | | | | Crystal |
|---|---|---|---|---|---|---|---|
| | Placement | Purity % | Purity % | Purity % | Purity % | Purity % | form |
| Sample | conditions | Onset | 1 month | 2 months | 3 months | 6 months | D |
| | 25° C., 60% RH | 97.9 | 97.8 | 97.8 | 97.8 | 97.8 | D |
| | 40° C., 75% RH | | 97.8 | 97.8 | 97.7 | 97.6 | D |

TABLE 28

| | | | | | | | Crystal |
|---|---|---|---|---|---|---|---|
| | Placement | Purity % | Purity % | Purity % | Purity % | Purity % | form |
| Sample | conditions | Onset | 1 month | 2 months | 3 months | 6 months | E |
| | 25° C., 60% RH | 97.9 | 97.9 | 97.8 | 97.8 | 97.8 | E |
| | 40° C., 75% RH | | 97.8 | 97.8 | 97.8 | 97.4 | E |

TABLE 29

| | | | | | | | Crystal |
|---|---|---|---|---|---|---|---|
| | Placement | Purity % | Purity % | Purity % | Purity % | Purity % | form |
| Sample | conditions | Onset | 1 month | 2 months | 3 months | 6 months | Q |
| | 25° C., 60% RH | 99.6 | 99.5 | 99.5 | 99.5 | 99.4 | Q |
| | 40° C., 75% RH | | 99.5 | 99.4 | 99.4 | 99.1 | Q |

TABLE 30

| | | | | | | | Crystal |
|---|---|---|---|---|---|---|---|
| | Placement | Purity % | Purity % | Purity % | Purity % | Purity % | form |
| Sample | conditions | Onset | 1 month | 2 months | 3 months | 6 months | U |
| | 25° C., 60% RH | 99.7 | 99.7 | 99.6 | 99.6 | 99.6 | U |
| | 40° C., 75% RH | | 99.7 | 99.6 | 99.6 | 99.5 | U |

Conclusion: The free crystal forms D, E, Q, and U demonstrate good physical and chemical stability when subjected to long-term accelerated stability conditions for a duration of six months.

What is claimed is:

1. A crystal form A, B, C, D, E, F, G, H, I, J, K, L, M, N, O, P, Q, U, X, Y, V, R, S or T of a compound of formula (I), 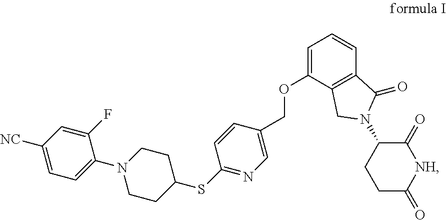 [SMILES: N#Cc1ccc(N2CCC(Sc3ccc(COc4cccc5c4CN([C@H]4CCC(=O)NC4=O)C5=O)cn3)CC2)c(F)c1]
wherein (1) the crystal form A has an X-ray powder diffraction pattern, represented by a diffraction angle 2θ, having characteristic peaks at 5.765, 8.061, 9.925, 16.632, 17.900, 19.469, and 21.115;
(2) the crystal form B has an X-ray powder diffraction pattern, represented by a diffraction angle 2θ, having characteristic peaks at 4.977, 6.788, 10.047, 14.143, 15.684, 18.547, and 20.840;
(3) the crystal form C has an X-ray powder diffraction pattern, represented by a diffraction angle 2θ, having characteristic peaks at 5.653, 7.974, 9.989, 16.143, 17.860, 18.992, and 20.972;
(4) the crystal form D has an X-ray powder diffraction pattern, represented by a diffraction angle 2θ, having characteristic peaks at 6.831, 9.845, 13.453, 18.225, 20.117, 20.891, and 23.006;
(5) the crystal form E has an X-ray powder diffraction pattern, represented by a diffraction angle 2θ, having characteristic peaks at 5.864, 7.573, 8.087, 10.003, 16.444, 19.349, and 20.553,
(6) the crystal form F has an X-ray powder diffraction pattern, represented by a diffraction angle 2θ, having characteristic peaks at 5.062, 7.820, 10.077, 14.231, 16.672, 18.586, and 20.435;
(7) the crystal form G has an X-ray powder diffraction pattern, represented by a diffraction angle 2θ, having characteristic peaks at 5.999, 7.972, 9.951, 11.388, 17.812, 20.975, and 25.819;
(8) the crystal form H has an X-ray powder diffraction pattern, represented by a diffraction angle 2θ, having characteristic peaks at 5.758, 7.533, 9.901, 14.267, 16.420, 18.103, and 26.356;
(9) the crystal form I has an X-ray powder diffraction pattern, represented by a diffraction angle 2θ, having characteristic peaks at 5.294, 6.826, 7.564, 10.739, 13.699, 16.812, and 20.709;
(10) the crystal form J has an X-ray powder diffraction pattern, represented by a diffraction angle 2θ, having characteristic peaks at 5.041, 10.068, 16.424, 20.544, 21.190, 24.077, and 25.433;
(11) the crystal form K has an X-ray powder diffraction pattern, represented by a diffraction angle 2θ, having characteristic peaks at 5.156, 7.699, 10.339, 14.334, 16.203, 18.327, and 23.418;
(12) the crystal form L has an X-ray powder diffraction pattern, represented by a diffraction angle 2θ, having characteristic peaks at 4.932, 5.360, 9.831, 14.844, 18.244, 20.104, and 24.914;
(13) the crystal form M has an X-ray powder diffraction pattern, represented by a diffraction angle 2θ, having characteristic peaks at 14.959, 16.322, 18.410, 20.748, 22.067, 23.670, and 26.863;
(14) the crystal form N has an X-ray powder diffraction pattern, represented by a diffraction angle 2θ, having characteristic peaks at 5.028, 9.942, 10.900, 15.428, 18.410, 20.274, and 25.252;
(15) the crystal form O has an X-ray powder diffraction pattern, represented by a diffraction angle 2θ, having characteristic peaks at 11.796, 17.423, 18.081, 19.136, 21.707, 22.165, and 25.719;
(16) the crystal form P has an X-ray powder diffraction pattern, represented by a diffraction angle 2θ, having characteristic peaks at 5.359, 7.491, 10.786, 14.249, 16.527, 17.729, and 20.798;
(17) the crystal form Q has an X-ray powder diffraction pattern, represented by a diffraction angle 2θ, having characteristic peaks at 5.038, 10.152, 15.850, 16.574, 18.892, 20.760, and 21.835;
(18) the crystal form U has an X-ray powder diffraction pattern, represented by a diffraction angle 2θ, having characteristic peaks at 14.155, 15.745, 17.314, 17.997, 18.838, 20.512, and 21.415;
(19) the crystal form X has an X-ray powder diffraction pattern, represented by a diffraction angle 2θ, having characteristic peaks at 6.864, 9.873, 10.963, 13.801, 16.089, 18.006, and 20.929;
(20) the crystal form Y has an X-ray powder diffraction pattern, represented by a diffraction angle 2θ, having characteristic peaks at 5.676, 7.666, 14.260, 16.562, 18.020, 21.802, and 26.425;
(21) the crystal form V has an X-ray powder diffraction pattern, represented by a diffraction angle 2θ, having characteristic peaks at 5.649, 6.154, 6.720, 11.651, 18.757, 19.813, and 23.948;
(22) the crystal form R has an X-ray powder diffraction pattern, represented by a diffraction angle 2θ, having characteristic peaks at 5.534, 7.611, 10.033, 15.782, 17.101, 19.017, and 20.567;
(23) the crystal form S has an X-ray powder diffraction pattern, represented by a diffraction angle 2θ, having characteristic peaks at 6.576, 9.082, 10.921, 13.592, 19.965, 21.403, and 24.207;
(24) the crystal form T has an X-ray powder diffraction pattern, represented by a diffraction angle 2θ, having characteristic peaks at 6.915, 9.177, 9.984, 11.012, 13.595, 16.156, and 20.138.

2-33. (canceled)

34. The crystal form A, B, C, D, E, F, G, H, I, J, K, L, M, N, O, P, Q, U, X, Y, V, R, S or T of the compound of formula (I) according to claim 1,
wherein (1) the crystal form A has an X-ray powder diffraction pattern, represented by a diffraction angle 2θ, having characteristic peaks at 5.765, 7.465, 8.061, 9.925, 12.890, 15.085, 16.632, 17.900, 19.469, and 21.115;
(2) the crystal form B has an X-ray powder diffraction pattern, represented by a diffraction angle 2θ, having characteristic peaks at 4.977, 6.788, 10.047, 14.143, 15.684, 18.547, 20.840, 24.096, and 25.505;
(3) the crystal form C has an X-ray powder diffraction pattern, represented by a diffraction angle 2θ, having characteristic peaks at 5.653, 7.974, 9.989, 11.505, 12.798, 14.265, 16.143, 17.860, 18.992, and 20.972;
(4) the crystal form D has an X-ray powder diffraction pattern, represented by a diffraction angle 2θ, having characteristic peaks at 6.831, 9.845, 10.927, 13.453, 16.096, 18.225, 20.117, 20.891, 23.006, and 26.132;
(5) the crystal form E has an X-ray powder diffraction pattern, represented by a diffraction angle 2θ, having characteristic peaks at 5.864, 7.573, 8.087, 10.003, 12.471, 15.165, 16.444, 17.432, 19.349, and 20.553;
(6) the crystal form F has an X-ray powder diffraction pattern, represented by a diffraction angle 2θ, having characteristic peaks at 5.062, 7.820, 10.077, 14.231, 15.192, 16.672, 18.586, 20.435, 21.868, and 25.442;
(7) the crystal form G has an X-ray powder diffraction pattern, represented by a diffraction angle 2θ, having characteristic peaks at 5.999, 7.396, 7.972, 8.637, 9.951, 11.388, 15.291, 17.812, 20.975, and 25.819;
(8) the crystal form H has an X-ray powder diffraction pattern, represented by a diffraction angle 2θ, having characteristic peaks at 5.758, 7.533, 9.901, 14.267, 16.420, 18.103, 18.917, 20.489, 24.049, and 26.356;
(9) the crystal form J has an X-ray powder diffraction pattern, represented by a diffraction angle 2θ, having characteristic peaks at 5.041, 8.212, 10.068, 14.101, 15.167, 16.424, 20.544, 21.190, 24.077, and 25.433;
(10) the crystal form K has an X-ray powder diffraction pattern, represented by a diffraction angle 2θ, having characteristic peaks at 5.156, 7.699, 10.339, 14.334, 16.203, 18.327, 23.418, 25.348, 25.919, and 26.446;
(11) the crystal form L has an X-ray powder diffraction pattern, represented by a diffraction angle 2θ, having characteristic peaks at 4.932, 5.360, 9.831, 10.753, 14.844, 16.369, 18.244, 20.104, 23.129, and 24.914;
(12) the crystal form M has an X-ray powder diffraction pattern, represented by a diffraction angle 2θ, having characteristic peaks at 14.959, 16.322, 18.410, 20.748, 22.067, 23.670, 24.839, 25.873, 26.863, and 27.811;
(13) the crystal form N has an X-ray powder diffraction pattern, represented by a diffraction angle 2θ, having characteristic peaks at 5.028, 7.671, 9.942, 10.900, 15.428, 16.560, 18.410, 20.274, 24.036, and 25.252;
(14) the crystal form O has an X-ray powder diffraction pattern, represented by a diffraction angle 2θ, having characteristic peaks at 7.843, 11.796, 17.423, 18.081, 19.136, 21.707, 22.165, 24.412, 25.719, and 28.521;
(15) the crystal form P has an X-ray powder diffraction pattern, represented by a diffraction angle 2θ, having characteristic peaks at 5.359, 7.491, 9.905, 10.786, 13.192, 14.249, 16.527, 17.729, 18.862, and 20.798;
(16) the crystal form Q has an X-ray powder diffraction pattern, represented by a diffraction angle 2θ, having characteristic peaks at 5.038, 10.152, 11.175, 15.850, 16.574, 18.892, 20.760, 21.835, 23.905, and 25.784;
(17) the crystal form U has an X-ray powder diffraction pattern, represented by a diffraction angle 2θ, having characteristic peaks at 14.155, 15.745, 16.564, 17.314, 17.997, 18.838, 20.512, 21.415, 23.557, and 26.313;
(18) the crystal form X has an X-ray powder diffraction pattern, represented by a diffraction angle 2θ, having characteristic peaks at 6.864, 9.873, 10.963, 13.801, 16.089, 18.006, 20.929, and 26.203;
(19) the crystal form Y has an X-ray powder diffraction pattern, represented by a diffraction angle 2θ, having characteristic peaks at 5.676, 7.666, 9.985, 12.634, 14.260, 16.562, 18.020, 21.802, 26.425, and 26.974;
(20) the crystal form V has an X-ray powder diffraction pattern, represented by a diffraction angle 2θ, having characteristic peaks at 5.649, 6.154, 6.720, 9.778, 11.651, 17.570, 18.757, 19.813, 23.948, and 26.995;
(21) the crystal form R has an X-ray powder diffraction pattern, represented by a diffraction angle 2θ, having characteristic peaks at 5.534, 7.611, 10.033, 11.857, 12.737, 15.782, 17.101, 19.017, 20.567, and 23.692;
(22) the crystal form S has an X-ray powder diffraction pattern, represented by a diffraction angle 2θ, having characteristic peaks at 6.576, 9.082, 10.921, 13.592, 16.805, 19.965, 21.403, 24.207, 25.662, and 27.457;
(23) the crystal form T has an X-ray powder diffraction pattern, represented by a diffraction angle 2θ, having characteristic peaks at 6.915, 9.177, 9.984, 11.012, 13.595, 15.174, 16.156, 20.138, 24.261, and 26.391.

35. The crystal form A, B, C, D, E, F, G, H, I, J, K, L, M, N, O, P, Q, U, X, Y, V, R, S or T of the compound of formula (I) according to claim 1,
wherein (1) the crystal form A has an X-ray powder diffraction pattern, represented by a diffraction angle 2θ, having characteristic peaks at 5.765, 7.465, 8.061, 9.925, 11.674, 12.890, 14.270, 15.085, 16.632, 17.900, 18.715, 19.469, and 21.115;
(2) the crystal form C has an X-ray powder diffraction pattern, represented by a diffraction angle 2θ, having characteristic peaks at 3.533, 5.653, 7.974, 8.790, 9.989, 11.505, 12.798, 14.265, 15.277, 16.143, 17.860, 18.992, and 20.972;
(3) the crystal form E has an X-ray powder diffraction pattern, represented by a diffraction angle 2θ, having characteristic peaks at 5.864, 7.573, 8.087, 10.003, 11.701, 12.471, 15.165, 16.444, 17.432, 19.349, 20.553, 21.067, and 21.709;
(4) the crystal form F has an X-ray powder diffraction pattern, represented by a diffraction angle 2θ, having characteristic peaks at 5.062, 7.820, 10.077, 14.231, 15.192, 16.672, 18.586, 20.435, 21.868, 24.193, 25.442, 26.303, and 28.629;
(5) the crystal form G has an X-ray powder diffraction pattern, represented by a diffraction angle 2θ, having characteristic peaks at 5.999, 7.396, 7.972, 8.637, 9.951, 11.388, 12.763, 15.291, 17.812, 20.975, 23.408, 25.819, and 27.400;
(6) the crystal form H has an X-ray powder diffraction pattern, represented by a diffraction angle 2θ, having characteristic peaks at 5.758, 7.533, 9.901, 14.267, 16.420, 18.103, 18.917, 20.489, 21.563, 24.049, and 26.356;
(7) the crystal form J has an X-ray powder diffraction pattern, represented by a diffraction angle 2θ, having characteristic peaks at 5.041, 8.212, 10.068, 14.101, 15.167, 16.424, 20.544, 21.190, 22.036, 22.679, 24.077, 25.433, and 26.454;
(8) the crystal form M has an X-ray powder diffraction pattern, represented by a diffraction angle 2θ, having characteristic peaks at 14.959, 16.322, 18.410, 20.748, 22.067, 23.670, 24.322, 24.839, 25.873, 26.863, and 27.811;
(9) the crystal form N has an X-ray powder diffraction pattern, represented by a diffraction angle 2θ, having characteristic peaks at 5.028, 7.671, 9.942, 10.900, 12.677, 15.428, 16.560, 18.410, 20.274, 24.036, 25.252, and 26.385;
(10) the crystal form O has an X-ray powder diffraction pattern, represented by a diffraction angle 2θ, having characteristic peaks at 7.843, 11.796, 15.455, 17.423, 18.081, 19.136, 21.055, 21.707, 22.165, 24.412, 25.719, 27.538, and 28.521;
(11) the crystal form P has an X-ray powder diffraction pattern, represented by a diffraction angle 2θ, having characteristic peaks at 5.359, 7.491, 9.905, 10.786, 13.192, 14.249, 16.527, 17.729, 18.862, 20.798, 23.799, and 26.555;
(12) the crystal form Q has an X-ray powder diffraction pattern, represented by a diffraction angle 2θ, having characteristic peaks at 5.038, 7.682, 10.152, 11.175, 14.218, 15.850, 16.574, 18.892, 20.760, 21.835, 23.905, 25.784, and 26.418;
(13) the crystal form U has an X-ray powder diffraction pattern, represented by a diffraction angle 2θ, having characteristic peaks at 7.657, 14.155, 15.745, 16.564, 17.314, 17.997, 18.838, 20.512, 21.415, 23.557, 25.711, 26.313, and 28.029;
(14) the crystal form Y has an X-ray powder diffraction pattern, represented by a diffraction angle 2θ, having characteristic peaks at 5.676, 7.666, 9.985, 12.634, 14.260, 16.562, 18.020, 21.802, 24.051, 25.846, 26.425, and 26.974;
(15) the crystal form V has an X-ray powder diffraction pattern, represented by a diffraction angle 2θ, having characteristic peaks at 5.649, 6.154, 6.720, 9.778, 11.651, 13.576, 17.570, 18.757, 19.813, 21.905, 23.948, 25.825, and 26.995;
(16) the crystal form R has an X-ray powder diffraction pattern, represented by a diffraction angle 2θ, having characteristic peaks at 5.534, 7.611, 10.033, 11.148, 11.857, 12.737, 14.179, 15.782, 17.101, 19.017, 20.567, 21.871, and 23.692;
(17) the crystal form S has an X-ray powder diffraction pattern, represented by a diffraction angle 2θ, having characteristic peaks at 6.576, 7.890, 9.082, 10.921, 13.592, 15.043, 16.805, 19.965, 21.403, 24.207, 25.662, 26.537, and 27.457;
(18) the crystal form T has an X-ray powder diffraction pattern, represented by a diffraction angle 2θ, having characteristic peaks at 6.915, 9.177, 9.984, 11.012, 13.595, 15.174, 16.156, 18.509, 20.138, 22.954, 24.261, 26.391, and 27.514.

36. The crystal form A, B, C, D, E, F, G, H, I, J, K, L, M, N, O, P, Q, U, X, Y, V, R, S or T of the compound of formula (I) according to claim 1,
wherein (1) the crystal form A has the X-ray powder diffraction pattern represented by the diffraction angle 2θ as shown in FIG. 2;
(2) the crystal form B has the X-ray powder diffraction pattern represented by the diffraction angle 2θ as shown in FIG. 3;
(3) the crystal form C has the X-ray powder diffraction pattern represented by the diffraction angle 2θ as shown in FIG. 4;
(4) the crystal form D has the X-ray powder diffraction pattern represented by the diffraction angle 2θ as shown in FIG. 5;
(5) the crystal form E has the X-ray powder diffraction pattern represented by the diffraction angle 2θ as shown in FIG. 6;
(6) the crystal form F has the X-ray powder diffraction pattern represented by the diffraction angle 2θ as shown in FIG. 7;
(7) the crystal form G has the X-ray powder diffraction pattern represented by the diffraction angle 2θ as shown in FIG. 8;
(8) the crystal form H has the X-ray powder diffraction pattern represented by the diffraction angle 2θ as shown in FIG. 9;
(9) the crystal form I has the X-ray powder diffraction pattern represented by the diffraction angle 2θ as shown in FIG. 10;
(10) the crystal form J has the X-ray powder diffraction pattern represented by the diffraction angle 2θ as shown in FIG. 11;
(11) the crystal form K has the X-ray powder diffraction pattern represented by the diffraction angle 2θ as shown in FIG. 12;
(12) the crystal form L has the X-ray powder diffraction pattern represented by the diffraction angle 2θ as shown in FIG. 13;
(13) the crystal form M has the X-ray powder diffraction pattern represented by the diffraction angle 2θ as shown in FIG. 14;
(14) the crystal form N has the X-ray powder diffraction pattern represented by the diffraction angle 2θ as shown in FIG. 15;
(15) the crystal form O has the X-ray powder diffraction pattern represented by the diffraction angle 2θ as shown in FIG. 16;
(16) the crystal form P has the X-ray powder diffraction pattern represented by the diffraction angle 2θ as shown in FIG. 17;
(17) the crystal form Q has the X-ray powder diffraction pattern represented by the diffraction angle 2θ as shown in FIG. 18;
(18) the crystal form U has the X-ray powder diffraction pattern represented by the diffraction angle 2θ as shown in FIG. 19;
(19) the crystal form X has the X-ray powder diffraction pattern represented by the diffraction angle 2θ as shown in FIG. 20;
(20) the crystal form Y has the X-ray powder diffraction pattern represented by the diffraction angle 2θ as shown in FIG. 21;
(21) the crystal form V has the X-ray powder diffraction pattern represented by the diffraction angle 2θ as shown in FIG. 22;
(22) the crystal form R has the X-ray powder diffraction pattern represented by the diffraction angle 2θ as shown in FIG. 23;
(23) the crystal form S has the X-ray powder diffraction pattern represented by the diffraction angle 2θ as shown in FIG. 24;
(24) the crystal form T has the X-ray powder diffraction pattern represented by the diffraction angle 2θ as shown in FIG. 25.

37. The crystal form according to claim 1, wherein 2θ angle has an error range of ±0.20.

38. A pharmaceutical composition comprising the crystal form according to claim 1.

39. The pharmaceutical composition according to claim 38, comprising a pharmaceutically acceptable carrier, a diluent, or an excipient.

40. A method for preparing a pharmaceutical composition, comprising the step of mixing the crystal form according to claim 1 with a pharmaceutically acceptable carrier, a diluent, or an excipient.

41. A method for treating or preventing a disease in a subject in need thereof, comprising administering an effective amount of the crystal form according to claim 1 to the subject, the disease is a cancer, an angiogenesis-related condition, pain, macular degeneration or related syndrome, a skin disease, a pulmonary disease, an asbestos-related disease, a parasitic disease, an immunodeficiency disease, a CNS disease, a CNS injury, atherosclerosis or related condition, sleep disorder or related condition, an infectious disease, hemoglobinopathy or related condition, or a TNFα-related condition.

42. The method according to claim 41, wherein the disease is a cancer or a CNS injury.

43. The method according to claim 41, wherein the cancer is selected from the group consisting of leukemia, myeloma, lymphoma, melanoma, skin cancer, liver cancer, kidney cancer, lung cancer, nasopharyngeal cancer, gastric cancer, esophageal cancer, colorectal cancer, gallbladder cancer, bile duct cancer, chorionic epithelioma, pancreatic cancer, polycythemia vera, pediatric tumor, cervical cancer, ovarian cancer, breast cancer, bladder cancer, urothelial cancer, ureteral tumor, prostate cancer, seminoma, testicular tumor, head and neck tumor, head and neck squamous cell carcinoma, endometrial cancer, thyroid cancer, sarcoma, osteoma, neuroblastoma, neuroendocrine cancer, brain tumor, CNS cancer, astrocytoma, and glioma.

44. The method according to claim 41, wherein the liver cancer is hepatocellular carcinoma; the colorectal cancer is colon cancer or rectal cancer; the sarcoma is osteosarcoma or soft tissue sarcoma; and the glioma is glioblastoma;
or, the myeloma is multiple myeloma (MM) and myelodysplastic syndrome (MDS).

45. The method according to claim 44, wherein the multiple myeloma is relapsed, refractory, or resistant.

46. The method according to claim 44, wherein the multiple myeloma is refractory or resistant to lenalidomide or pomalidomide.

47. A method for treating or preventing a disease in a subject in need thereof, comprising administering an effective amount of the pharmaceutical composition according to claim 38 to the subject, the disease is a cancer, an angiogenesis-related condition, pain, macular degeneration or related syndrome, a skin disease, a pulmonary disease, an asbestos-related disease, a parasitic disease, an immunodeficiency disease, a CNS disease, a CNS injury, atherosclerosis or related condition, sleep disorder or related condition, an infectious disease, hemoglobinopathy or related condition, or a TNFα-related condition.

48. The method according to claim 47, wherein the disease is a cancer or a CNS injury.

49. The method according to claim 47, wherein the cancer is selected from the group consisting of leukemia, myeloma, lymphoma, melanoma, skin cancer, liver cancer, kidney cancer, lung cancer, nasopharyngeal cancer, gastric cancer, esophageal cancer, colorectal cancer, gallbladder cancer, bile duct cancer, chorionic epithelioma, pancreatic cancer, polycythemia vera, pediatric tumor, cervical cancer, ovarian cancer, breast cancer, bladder cancer, urothelial cancer, ureteral tumor, prostate cancer, seminoma, testicular tumor, head and neck tumor, head and neck squamous cell carcinoma, endometrial cancer, thyroid cancer, sarcoma, osteoma, neuroblastoma, neuroendocrine cancer, brain tumor, CNS cancer, astrocytoma, and glioma.

50. The method according to claim 47, wherein the liver cancer is hepatocellular carcinoma; the colorectal cancer is colon cancer or rectal cancer; the sarcoma is osteosarcoma or soft tissue sarcoma; and the glioma is glioblastoma;
or, the myeloma is multiple myeloma (MM) and myelodysplastic syndrome (MDS).

51. The method according to claim 50, wherein the multiple myeloma is relapsed, refractory, or resistant.

52. The method according to claim 50, wherein the multiple myeloma is refractory or resistant to lenalidomide or pomalidomide.
